(19) United States
(12) Patent Application Publication
(10) Pub. No.: US 2009/0182127 A1
(43) Pub. Date: Jul. 16, 2009

(54) Production of Bispecific Antibodies

(75) Inventors: Kristian Kjaergaard, Ballerup (DK); Jens Jacob Hansen, Jyllinge (DK); Soren Berg Padkaer, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/305,669

(22) PCT Filed: Jun. 22, 2007
(86) PCT No.: PCT/EP07/56280

§ 371 Date: Jan. 20, 2009

Related U.S. Application Data
(60) Provisional application No. 60/816,773, filed on Jun. 27, 2006.

(30) Foreign Application Priority Data
Jun. 22, 2006 (EP) 06115898.6

(51) Int. Cl. C07K 16/46; C07K 1/00

(52) U.S. Cl. 530/387.3; 530/402

(57) ABSTRACT

Bispecific antibodies comprising (a) a first light-heavy chain pair having specificity for a first target and a sufficient number of substitutions in its heavy chain constant domain with respect to a corresponding wild-type antibody of the same isotype to significantly reduce the formation of first heavy chain-first heavy chain dimers and (b) a second light-heavy chain pair comprising a heavy chain having a sequence that is complementary to the sequence of the first pair heavy chain sequence with respect to the formation of intramolecular ionic interactions, wherein the first pair or second pair comprises a substitution in the light chain and complementary substitution in the heavy chain that reduces the ability of the light chain to interact with the heavy chain of the other light chain-heavy chain pair are provided. Methods of producing such antibodies in one or more cells also are provided.

### FIELD OF THE INVENTION

The various aspects of the invention described herein relate to methods for the production of bispecific antibodies, bispecific antibody molecules produced by these and other methods, and related compositions and methods.

### BACKGROUND OF THE INVENTION

Antibodies (or “immunoglobulins”) are proteins secreted by mammalian (e.g., human) B lymphocyte-derived plasma cells in response to the appearance of an antigen. The basic unit of each antibody is a monomer. An antibody molecule can be monomeric, dimeric, trimeric, tetrameric, pentameric, etc. The antibody monomer is a “Y”-shaped molecule that consists of two identical heavy chains and two identical light chains.

Specifically, each such antibody monomer contains a pair of identical heavy chains (HCs) and a pair of identical light chains (LCs). Each LC has one variable domain (VL) and one constant domain (CL), while each HC has one variable (VH) and three constant domains (CH1, CH2, and CH3). The CH1 and CH2 domains are connected by a hinge region. Each polypeptide is characterized by a number of intrachain disulphide bridges and polypeptides are interconnected by additional disulphide bridges. In addition to disulphide bridging the polypeptides, the polypeptide chains also are associated due to ionic interactions (which interactions are directly relevant to many aspects of the invention described herein).

There are five types of heavy chain: γ, δ, α, μ and ε (or G, D, A, M, and E). They define classes of immunoglobulins. H chains of all isotypes associate with light (L) chains of two isotypes—k and l. Thus, the basic H₂L₂ composition of an antibody can be specified in terms of its H and L isotypes; e.g., e₂k₂, (m₂l₂)₅, etc. Based on the differences in their heavy chains, immunoglobulin molecules are divided into five major classes: IgG, IgM, IgA, IgE, and IgD. Immunoglobulin G (“IgG”) is the predominant immunoglobulin of internal components such as blood, cerebrospinal fluid and peritoneal fluid (fluid present in the abdominal cavity). IgG is the only class of immunoglobulin that crosses the placenta, conferring the mother's immunity on the fetus. IgG makes up 80% of the total immunoglobulins. It is the smallest immunoglobulin, with a molecular weight of 150,000 Daltons. Thus it can readily diffuse out of the body's circulation into the tissues. All currently approved antibody drugs comprise IgG or IgG-derived molecules.

In some species, the immunoglobulin classes are further differentiated according to subclasses, adding another layer of complexity to antibody structure. In humans, for example, IgG antibodies comprise four IgG subclasses—IgG1, IgG2, IgG3, and IgG4. Each subclass corresponds to a different heavy chain isotype, designated g1 (IgG1), g2 (IgG2), g3 (IgG3), g4 (IgG4), a1 (IgA1) or a2 (IgA2).

The production of antibody molecules, by various means, is generally well understood. U.S. Pat. No. 6,331,415 (Cabilly et al.), for example, describes a method for the recombinant production of immunoglobulin where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of E. coli. Various other techniques relevant to the production of antibodies are described in, e.g., Harlow, et al., ANTIBODIES: A LABORATORY MANUAL, Cold Spring Harbor Laboratory Press, Cold Spring Harbor, N.Y., (1988) and WO2006028936.

In mammals (and certain other chordates), the reaction between antibodies and an antigen (which is usually associated with an infectious agent) leads to elimination of the antigen and its source. This reaction is highly specific, that is, a particular antibody usually reacts with only one type of antigen. The antibody molecules do not destroy the infectious agent directly, but, rather, “tag” the agent for destruction by other components of the immune system. In mammals such as humans, the tag is constituted by the CH2-CH3 part of the antibody, commonly referred to as the Fc domain.

Bispecific antibodies (BsAbs), with affinity towards two independent antigens, have been previously described (reviewed by Holliger and Winter 1993 Curr. Opin. Biotech. 4, 446-449 (see also Poljak, R. J., et al. (1994) Structure 2:1121-1123; and Cao et al. (1998), Bioconjugate Chem. 9, 635-644)). Such antibodies may be particularly useful in (among other things) redirection of cytotoxic agents or immune effector cells to target sites, as tumors. To date, most bispecific antibodies have been created by connecting VH and VL domains of two independent antibodies using a linker that is too short to allow pairing between domains on the same chain, thus driving the pairing between complementary domains on different chains to recreate the two antigen-binding sites. A major drawback for this type of antibody molecule is the lack of the Fc domain and thus the ability of the antibody to trigger an effector function (e.g. complement activation, Fc-receptor binding etc.).

“Full length” bi-specific antibodies (BsAb-IgG) (BsAbs comprising a functional antibody Fc domain) also have previously been created, typically by chemical cross-linking of two different IgG molecules (Zhu et al 1994 Cancer Lett., 86, 127-134) or co-expressing two immunoglobulin G molecules (“IgGs”) in hybrid hybridomas (Suresh et al 1986 Methods Enzymol 121, 210-228). Chemical cross-linking, however, is often inefficient and can lead to loss of antibody activity. Coexpression of two different IgGs in a hybrid hybridoma may produce up to 10 different heavy- and light-chain pairs, hence compromising the yield of BsAb-IgG (see, e.g., US Patent Application 2003/007835). In both methods, purification of the BsAb-IgG from non-functional species, such as multimeric aggregates resulting from chemical modification and homodimers of heavy or light chains and non-cognate heavy-light chain pairs, is often difficult and the yield is usually low.

US Patent Application 20030078385 (Arathoon et al.—Genentech) describes a method of producing a multispecific antibody involving introducing (a) a specific and complementary interaction “at the interface of a first polypeptide and the interface of a second polypeptide,” by creating “protuberance-into-cavity” complementary regions (by replacement of amino acids with smaller side chains with those of larger chains or visa versa) so as to promote heteromultimer formation and hinder homomultimer formation; and/or (b) a free thiol-containing residue at the interface of a first polypeptide and a corresponding free thiol-containing residue in the interface of a second polypeptide, such that a non-naturally occurring disulfide bond is formed between the first and second polypeptide. The '385 application also describes generating complementary hydrophobic and hydrophilic regions in the multimerization domain (a portion of the constant domain comprising the C_{H3} interface). The methods of the '385 application call for use of a single (“common”) variable light chain. Such “knobs-into-holes” with common light chain bispecific antibodies, and other types of bispecific antibodies (and methods used to such produce bispecific antibodies) are reviewed in Marvin and Zhu, Acta Pharmacologica Sincia, 26(6):649-658 (2005) (see also Kontermann, Acta Pharacol. Sin., 26:1-9 (2005)).

There remains a need for alternative types of bispecific antibody molecules and methods of producing bispecific antibodies. The invention described herein provide such molecules and methods. These and other aspects and advantages of the invention will be apparent from the description of the invention provided herein.

### SUMMARY OF THE INVENTION

The invention described herein provides new bispecific antibodies, new methods for producing bispecific antibodies, and other various related methods and compositions.

In one exemplary aspect, the invention provides a bispecific antibody comprising (a) a first light-heavy chain pair having specificity for a first target and a sufficient number of substitutions in its heavy chain constant domain with respect to a corresponding wild-type antibody of the same isotype to significantly reduce the formation of first heavy chain-first heavy chain dimers and (b) a second light-heavy chain pair comprising a heavy chain having a sequence that is complementary to the sequence of the first pair heavy chain sequence with respect to the formation of intramolecular ionic interactions, wherein the first pair or second pair comprises a substitution in the light chain and complementary substitution in the heavy chain that reduces the ability of the light chain to interact with the heavy chain of the other light chain-heavy chain pair are provided. Methods of producing such antibodies in one or more cells also are provided.

These aspects of the invention are more fully described in, and additional aspects, features and advantages of the invention will become apparent upon reading, the description of the invention provided herein.

### DESCRIPTION OF THE DRAWINGS

FIG. 1: Schematic illustration of the ionic interactions between amino acids present in the constant domains of immunoglobulins.

FIG. 2: Schematic illustration of exemplary processes to generate bispecific antibodies by ex vivo assembly of individual antibody chains produced in various cells.

FIG. 3: Alignment of the constant part of the heavy chain for the KM and GM allotypes of IgG1.

FIG. 4: Alignment and labeling of the Kappa and Lambda constant regions of IgG1.

FIG. 5: A molecular surface illustration, showing the interaction points of one CH3 surface.

FIG. 6A-C: Alignment of immunoglobulin amino acid sequences from Human, Mouse, and Rat. The alignment demonstrates that regions in which ionic interaction pairs are present in a species are highly conserved, reflecting the applicability of the inventive methods in immunoglobulins derived from various species.

FIG. 7: Western blot using goat-anti-human Fc-HRP specific antibodies on supernatant from HEK293 6E cells 6 days after transfection with IgG1 heavy chain mutants lacking cysteine residues (Cys-Ala) in the hinge region. Lane 1: MagicMarker, Lane 2: TF-HC1-IgG1-Cys-Ala, Lane 3: KIR-HC2-IgG1-Cys-Ala, Lane 4: Untransfected cells.

FIG. 8: Western blot using Sheep-anti-human IgG1 primary antibody (The Binding Site AP006) and Rabbit-anti-Sheep HRP secondary antibody (DAKO 0163) on supernatant from HEK293 6E cells 6 days after transfection with the following: an anti-tissue factor (“TF”) antibody light chain/heavy chain IgG1 antibody pair that immunoreacts with human tissue factor (TF) to inhibit the binding of coagulation factor VIIa (FVIIa) (“TF-LC1+TF-HC1-IgG1” (similar abbreviations are used throughout)) (lane 1); anti-tissue factor/anti-KIR antibody light chain/heavy chain IgG1 antibody pair TF-LC1+anti-KIR (antibody pair that binds KIR2DL1 (Killer immunoglobulin like inhibitory receptor) KIR2DL2, and KIR2DL3 (“KIR”)-HC2-IgG1 (lane 2); anti-KIR/anti-TF light chain/heavy chain pair KIR-LC2+TF-HC1-IgG1 (lane 3); anti-KIR light chain/heavy chain pair KIR-LC2+KIR-HC2-IgG1 (lane 4); anti-TF/anti-KIR bispecific antibody TF-LC1+TF-HC1-IgG1+KIR-LC2+KIR-HC2-IgG1 (lane 5); TF-HC1-IgG1 (lane 6); KIR-HC2-IgG1 (lane 7); TF-HC1-IgG1+KIR-HC2-IgG1 (lane 8); and MagicMark™ XP (lane 9).

FIG. 9: Western blot using Goat-anti-human IgG1 kappa light chain primary antibody (Biosite 11904-35z) and Rabbit-anti-Goat HRP secondary antibody (DAKO Po160) on supernatant from HEK293 6E cells 6 days after transfection with: TF-LC1+TF-HC1-IgG1 (lane 1), TF-LC1+KIR-HC2-IgG1 (lane 2), KIR-LC2+TF-HC1-IgG1 (lane 3), KIR-LC2+KIR-HC2-IgG1 (lane 4), TF-LC1+TF-HC1-IgG1+KIR-LC2+KIR-HC2-IgG1 (lane 5), TF-HC1-IgG1 (lane 6), KIR-HC2-IgG1 (lane 7), TF-HC1-IgG1+KIR-HC2-IgG1 (lane 8), and MagicMark™ XP (lane 9). Rainbow marker (lane 1) and MagicMark™ XP (lane 10) also are shown.

FIG. 10: Binding of test antibody to immobilized anti-Ig followed by binding of human TF. Abbreviations: LC1 HC1=TF-LC1+TF-HC1-IgG1, LC2HC2=KIR-LC2+KIR-HC2-IgG1, Bispec=TF-LC1+TF-HC1-IgG1+KIR-LC2+KIR-HC2-IgG2.

FIG. 11: Binding of test antibody to immobilized human KIR2DL3 followed by binding to human TF. Abbreviations: LC1 HC1=TF-LC1+TF-HC1-IgG1, LC2HC2=KIR-LC2+KIR-HC2-IgG1, Bispec=TF-LC1+TF-HC1-IgG1+KIR-LC2+KIR-HC2-IgG2.

FIG. 12: The human TF binding part of the previous figure, normalized. Abbreviations: LC1 HC1=TF-LC1+TF-HC1-IgG1, LC2HC2=KIR-LC2+KIR-HC2-IgG1, Bispec=TF-LC1+TF-HC1-IgG1+KIR-LC2+KIR-HC2-IgG2.

FIG. 13: (A) Western blot using goat-anti-human IgG Fc specific-HRP antibody on supernatant from HEK293 6E cells 6 days after transfection. Lane 1: HC1-IgG1-Fc (unreduced), lane 2: HC1-IgG1-Fc (reduced), lane 3: HC2-IgG1-Fc (unreduced), lane 4: HC2-IgG1-Fc (reduced), lane 5: HC1-IgG1-Fc+HC2-IgG1-Fc (unreduced), lane 6: HC1-IgG1-Fc+HC2-IgG1-Fc (reduced). (B) Western blot using goat-anti-human IgG Fc specific-HRP anti-body on supernatant from HEK293 6E cells 6 days after transfection. Lane 1: HC1-IgG4-Fc (unreduced), lane 2: HC1-IgG4-Fc (reduced), lane 3: HC2-IgG4-Fc (unreduced), lane 4: HC2-IgG4-Fc (reduced), lane 5: HC1-IgG4-Fc+HC2-IgG4-Fc (unreduced), lane 6: HC1-IgG4-Fc+HC2-IgG4-Fc (reduced).

FIG. 14: Quantification of dimerization of IgG4 heavy chain mutants analyzer using Agilent 2100 Bioanalyzer. Supernatants from transiently expressed HEK293 6E cells were analyzed 6 days after transfection. The figure shows electrophoresis of protein bands corresponding to lane 1. Marker, Lane 2. Full length IgG4 control antibody, Lane 3. HC1-IgG4-Fc, Lane 4. HC2-IgG4-Fc, Lane 5. HC1-IgG4-Fc+HC2-IgG4-Fc.

FIG. 15: Electropherograms showing the protein quantity in FIG. 14 lanes 2-5, (A) to (D), respectively.

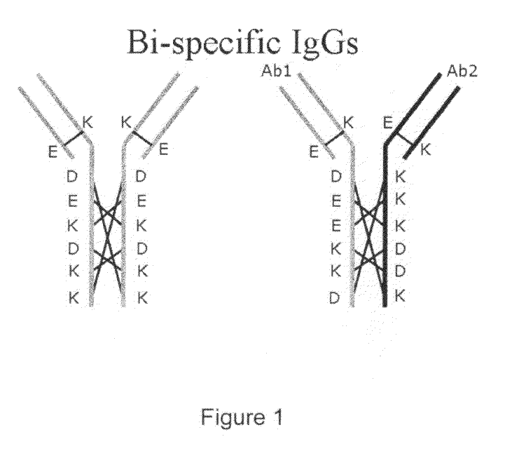

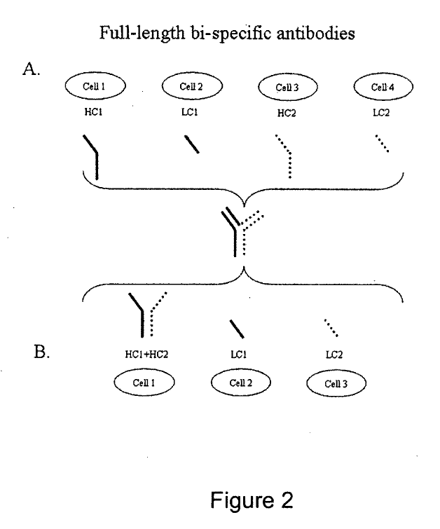

### DESCRIPTION OF THE INVENTION

The invention described herein arises, in part, from the inventors' discovery that pairs of amino acids in the constant domains of antibody monomers are significantly involved in the multimerization and stability of such antibody monomers (and antibody molecules as a whole in the case of antibody molecules such as IgG molecules) and can, accordingly, be modified by various methods, so as to better promote the formation of bispecific antibody monomers or molecules. Typically, such pairs of amino acids are primarily found in the heavy chains of antibody molecules (e.g., between certain amino acid residues present in the CH1 and CH3 constant regions of an IgG molecule). However, in some cases, as exemplified herein, heavy chain-light chain (CL) constant domain amino acid residue intramolecular ionic interactions also can be important to the formation of antibodies.

For example, in human immunoglobulin G antibodies (IgG Abs), the inventors have now discovered that ionic forces, which contribute to cross-linking the two heavy chain (“HC”) polypeptides of the tetrameric antibody molecule, are contributed mainly by six amino acids present in the CH3 region of the antibody in the following manner: E240-K253, D282-K292, and K322-D239 (sequence position numbers refer to the amino acid starting from the beginning of CH1 (according to UNIPROT-ID:IGHG1_HUMAN).

Using this discovery, the inventors have further discovered that, for example, by substituting HC amino acids of an IgG antibody (Ab1) with an affinity towards a first antigen (X) as follows—K253E, D282K, and K322D, it is possible to significantly reduce the self pairing of the human IgG Ab HC polypeptide (which normally occurs in the corresponding wild-type tetrameric antibody molecule). By similarly modifying the HC sequence of a second IgG antibody (Ab2), preferably with an affinity towards a second target (Y) by the substitutions D239K, E240K, and K292D, dimerization of such Ab2 HC polypeptides also is abolished.

In a similar fashion, the inventors have discovered that amino acids in position 15 of the CL of human Abs (numbering according to UNIPROT-ID:KAC_HUMAN) and K96 of CH1 normally form an ionic interaction between the light chain (LC) and HC of human IgG antibodies, bringing the two chains in sufficient proximity for sulfide-bridge formation between cysteine residues present in the LC (C105) and HC (C103) hinge regions. The inventors have further discovered that changing the amino acid residue at this position in one of the LCs (of Ab1 and Ab2) and cognate HC in the following manner, E15K on the LC and K96E on the HC, can prevent the modified LC from pairing with a non-cognate HC (e.g., if Ab1 is so modified, the Ab2 LC will not be able to associate with the Ab1 HC as readily as it would without such a modification).

The inventors have additionally discovered that co-expressing the polypeptides from these two modified antibodies can “restore” such ionic interactions that stabilize a human tetrameric antibody (e.g., E240-K253, D282-K292, and K322-D239) and pairing of the polypeptides, resulting in generation of a bi-specific antibody with an affinity towards different targets. Table 1 summarizes (in exemplary fashion) these various substitutions:

TABLE 1
Amino acid substitution in constant
domains of human IgG1 or IgG4.

| | |
|---|---|
| Antibody 1 | Antibody 2 |

CH3 mutations

| | |
|---|---|
| K253E | D239K |
| D282K | E240K |
| K322D | K292D |

CH1 mutations

K96E

CL mutations

E15K

The inventors have used such particular findings to invent new methods of producing antibodies and new antibody molecules, which expand upon and/or further define the specific discoveries described above.

In one such exemplary aspect, the invention described herein generally provides a new method for producing various types of bispecific antibodies.

This inventive method generally includes a step of identifying pairs of amino acid residues involved in constant domain intramolecular ionic interactions in an antibody molecule. Such ionic pair interaction residues (or “IPIRs”) can be identified by any suitable method. In one exemplary method, IPIRs are identified by generating or providing X-ray structures for light chain-heavy chain constant domain region interactions to identify IPIRs by identifying residues matching a set of criteria (e.g., propensity to engage in ionic interactions, availability to form such interactions, proximity to a potential partner residue, etc.), which may conveniently done by analyzing such structures or related sequences with a computer software program, such as the MOE (Molecular Operating Environment) software available from Chemical Computing Group (www.chemcomp.com).

It may be often the case that the identification of IPIRs in an antibody molecule can be extrapolated or correlated to similar antibody molecules (antibodies having identical constant domains by virtue of being from the same species or even a highly similar constant domain in terms of amino acid sequence identity). Constant domain ionic interactions identified in a particular type of antibody molecule of a particular species will likely always be identical for other antibodies of a same isotype in that species (e.g., IPIRs identified in a particular human immunoglobulin G (“IgG”) molecule will likely always be found in other human IgGs). Moreover, constant domain ionic interactions in an antibody of a particular isotype in one species will be readily translatable (if not identical) to antibody molecules of a similar isotype in other species having similar types of antibody molecules. For example, in humans, rats, and mice, antibody constant domain sequences exhibit greater than 90% sequence identity, such that IPIRs identified in one of these organisms will likely be identical or very similar to IPIRs in another one of these organisms. Thus, the step of identifying IPIRs in a particular antibody, in the above-described step, can be substituted by identifying IPIRs in a “type” of antibody, wherein “type” of antibody molecule refers to the isotype of the antibody molecule and either (a) the species origin of the antibody (or antibody's constant domain) or (b) an antibody of a different species but having a highly similar constant domain.

The inventive method further comprises preparing a first pair of antibody light chain and heavy chain proteins (which may be referred to as the “first light chain-heavy chain pair” or “FLCHCP”), which (a) has specificity for a first target (by virtue of the particular variable domains comprised therein) and (b) comprises a constant domain comprising at least some substitutions of amino acid residues normally involved in constant chain intramolecular interactions in a wild-type homolog or in the same “type” of antibody. The method also comprises preparing a second light chain-heavy chain pair (“SLCHCP”) having specificity for a second target and comprising a constant domain that comprises an amino acid sequence complementary to the FLCHCP pair in terms of constant domain intramolecular ionic interactions. The constant domain sequences are “complementary,” in that the substitutions in the first pair constant domain and second pair constant domain maximize ionic interactions between the first and second pairs with respect to “self” interactions (i.e., first pair:first pair or second pair:second pair interactions). In other words, the FLCHCP and SLCHCP collectively comprise substitution of a sufficient number of the amino acid residues normally involved in wild-type antibody (or antibody monomer) intramolecular interactions (e.g., in a wild-type homolog), such that bispecific tetrameric antibody molecules comprising both a FLCHCP and a SLCHCP (i.e., FLCHCP:SLCHP heteromultimers) form more frequently than monospecific tetramers (e.g., FLCHP:FLCHP or SLCHP:SLCHP homomultimers) when the FLCHCP and SLCHCP proteins are permitted to fold and associate (i.e., to form such multimers). The method furthermore includes mixing or otherwise contacting the FLCHCP and SLCHCP proteins under conditions suitable for folding and association of the various component chains to obtain such a tetrameric bispecific antibody. The specific parameters for this final step for any particular bispecific antibody so generated can be readily determined by ordinarily skilled artisans using no more than routine experimentation. Additional guidance in this respect is provided, and such parameters exemplified, elsewhere herein.

The invention also provides novel bispecific antibodies comprising a FLCHCP and a SLCHCP as described in the foregoing method. The FLCHCP and SLCHCP components of the BsAbs provided by the invention generally can have any suitable composition, so long as they meet the criteria described above (i.e., having sufficient variable domains and framework regions so as to provide a functionally bispecific antibody and having a sufficient constant domains (i.e., a sufficient portion of an Fc region) so as to comprise a number of IPIR-relevant substitutions (e.g., 5, 6, 7, 8, or 9 of such substitutions)). Typically, such bispecific antibodies can be characterized as lacking additional immunoglobulin molecules or fragments joined via covalent bonding by covalent linkage or expression as a fusion protein (e.g., as distinguished form, e.g., a so-called “tandem antibody,” diabody, tandem diabody, scFv-IgG fusion, etc.); however, in other aspects it is contemplated that bispecific antibodies of the invention may be linked or fused with other antibody molecules or fragments. In a particular aspect, the invention provides such an antibody (i.e., a bispecific antibody comprising a FLCHCP and a SLCHCP as described above), wherein the antibody comprises IPIR-relevant substitutions outside of, as well as optionally within, the antibody multimerization domain. In another particular aspect, the invention provides such an antibody wherein the antibody also or alternatively can be characterized by comprising a significant portion (e.g., at least about 50%, at least about 60%, at least about 70%, at least about 80%, at least about 85%, at least about 90%, at least about 95%, or more) of the Fc domain (of the nearest related or parent antibodies—e.g., of an IgG1 in the case of a BsAb of the invention derived from IgG1 sequences). In a more particular facet of this aspect (where the BsAb comprises a significant proportion of the Fc domain), the significant portion of the Fc domain is of sufficient size and composition that it imparts greater protein stability than compared to a substantially similar bispecific antibody lacking most or all of the Fc domain. In another more particular facet of this aspect, the portion of the Fc domain is of sufficient size and composition that it increases the in vivo half-life of the bispecific antibody (e.g., due to slower clearance from the circulation) as compared to a substantially similar bispecific antibody lacking the Fc domain; in still another particular aspect the portion of the Fc domain is functional (i.e., imparts antibody effector function to the bispecific antibody)). In other aspects, antibodies of the invention can be characterized by (in addition or alternatively to any of the other features described here) comprising a full length or near full length Fc domain that is not functional (e.g., by introduction of mutations into the Fc domain, derivatization of the Fc domain, or, typically, by expression of the antibody in a bacterial cell or other cell that is not capable of properly glycosylating the Fc domain). In yet another particular aspect, the invention provides a BsAb having a FLCHCP and a SLCHCP as described above, wherein, in addition to any or all of the foregoing (or following) described possible defining characteristics (e.g., possession of a significant proportion of an Fc domain as defined by any of the above-described facets, lacking additional conjugated Ig molecules, or both), or alternatively thereto, the BsAb comprises different first and second light chains (i.e., the first pair and second pair comprise significantly different light chains). In still another particular aspect, the invention provides a BsAb having a FLCHCP and a SLCHCP as described above wherein, in addition to any or all of the foregoing (or following) characteristics, or alternatively thereto, the BsAb lacks any non-naturally occurring cysteine-cysteine interactions (i.e., no modifications are made to the sequence(s) of the first and/or second pair to introduce additional cysteine-cysteine interactions in the antibody). In still another additional particular aspect, the invention provides a BsAb having a FLCHCP and a SLCHCP as described above, wherein, in addition to any or all of the foregoing (or following) characteristics, or alternatively thereto, the antibody is characterized by substantially or entirely lacking any modifications that would introduce protuberances and/or cavities into the multimerization domain (with respect to a wild-type homolog) (i.e., lacks artificial “knobs-into-holes” associations). In a further particular aspect, the invention provides a BsAb having a FLCHCP and a SLCHCP as described above wherein, in addition to any or all of the foregoing (or following) characteristics, or alternatively thereto, the antibody is characterized by the lack of any introduced hydrophobic or hydrophilic regions (particularly by introduction of more than 2, 3, 4, or 5 contiguous amino acid residues into any chain) in the multimerization domain (with respect to a wild-type homolog). In a further particular aspect, the invention provides a BsAb having a FLCHCP and a SLCHCP as described above wherein, in addition to any or all of the foregoing (or following) characteristics, or alternatively thereto, the antibody is characterized by the lack of any artificial linker between the VH and VL domains.

Any of these characteristics of such BsAb molecules (or any suitable combination thereof) may similarly characterize the production of BsAbs according to the aforementioned method (i.e., such methods are a feature of the invention—e.g., a method as described above wherein antibodies are produced without introducing any “knobs-into-holes” substitutions, new cysteine-cysteine disulfide bridges, and/or VH-VL linkers, etc.) and/or with different light chains in the FLCHCP and SLCHCP.

As exemplified by BsAbs of the invention characterized by possession of a full-length or near full length Fc domain, the BsAbs of the invention can be of any suitable size, provided that the antibody provides the required specific binding for the two different targets of interest and can include a sufficient number of IPIR-related modifications to provide for improved formation of the bispecific antibody with respect to “contaminant” antibody molecules. The description, “full length”, in this respect, refers to an antibody of similar size to a referenced wild-type immunoglobulin (e.g., an IgG). The phrase “near full length” refers to an antibody comprising nearly all of the Fc domain and other domains of a wild-type antibody molecule. Both types of BsAbs (amongst others) are provided by the present invention. In an advantageous aspect, antibodies of the invention can be characterized by comprising heavy chains that comprise at least the variable region, the first constant domain, the hinge region, the second constant domain, and third constant domain of an IgG. Typically, antibodies of the invention will comprise a significant portion of an antibody Fc domain. In other aspects, however, the heavy chain comprises only a portion of the CH1, CH2, and/or CH3 domains.

In a particular exemplary aspect, the invention provides a bispecific antibody comprising (a) a FLCHCP derived from a human antibody but comprising the following substitutions: K253E (i.e., the Lys residue present in the wild-type homolog constant region is substituted with a Glu residue), D282K, and K322D (unless otherwise specified, references to heavy chain amino acid residues herein are made with respect to the beginning of CH1 based on (according to UNIPROT-ID:IGHG1_HUMAN)); and (b) a SLCHCP derived from a human antibody but comprising substitutions D239K, E240K, and K292D, wherein either the FLCHCP or the SLCHCP comprises a light chain having the substitution E15K (unless otherwise specified, citations of light chain amino acid residue positions herein are made with reference to UNIPROT-ID:KAC_HUMAN) and a heavy chain comprising the substitution K96E (the other LCHCP being unmodified at these positions). The phrase “derived from an antibody,” herein, is used to refer to an antibody molecule or fragment that is identical or highly similar in terms of amino acid sequence composition (e.g., at least about 80%, at least about 85%, at least about 90%, at least about 95%, 96%, 97%, 98%, or 99% identical) to a reference (or “parent”) antibody or antibody-like molecule, other than the indicated (and possibly some number of unspecified additional) changes (e.g., the above-described specific substitutions). The phrase “derived from” is, in this sense, not intended to indicate (or limit) the method by which such an antibody or antibody fragment is generated (which may be by any suitable available method, such as recombinant expression, chemical protein synthesis, etc.). Given that a bispecific antibody of the invention may vary in composition from a wild-type antibody (due to insertions or deletions of one or several residues in the light chain(s), heavy chain(s), or light chain(s) and heavy chain(s)), references to positions used to identify substitutions in the bispecific antibody in respect of a parent antibody (or antibody sequence) are to be understood as referring to the amino acid residue(s) that most nearly corresponds with the indicated reference (e.g., wild-type parent antibody) residue (e.g., position 239 in the wild-type antibody, as described above, may correspond to position 237, 238, 240, or 241 in the bispecific antibody). An ordinarily skilled artisan will be able to determine what residues correspond to the indicated wild-type residues in such situations by using routine methods, such as by determining the optimal alignment for the amino acid sequences at issue (taking into consideration structural and other relevant data).

“Identity,” in the context of comparing amino acid sequences, can be determined by any suitable technique, such as (and as one suitable selection in the context of this invention) by employing a Needleman-Wunsch alignment analysis (see Needleman and Wunsch, J. Mol. Biol. (1970) 48:443-453), such as is provided via analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available, e.g., through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs). Further related methods for assessing identity are described in, e.g., International Patent Application WO 03/048185. The Gotoh algorithm, which seeks to improve upon the Needleman-Wunsch algorithm, alternatively can be used for global sequence alignments. See, e.g., Gotoh, J. Mol. Biol. 162:705-708 (1982).

In one advantageous aspect, bispecific antibodies of the invention are derived from human immunoglobulin G molecules. In general, bispecific antibodies of the invention can be generated from any suitable type of IgG molecule. In one advantageous aspect of the invention, the bispecific antibody is derived from a human IgG1. In another advantageous aspect, the bispecific antibody of the invention is derived from a human IgG4. In other aspects, the bispecific antibody is derived from a non-human (e.g., a primate or rodent) IgG molecule (or antibody type that is recognized as being substantially similar to a human IgG in terms of composition) (e.g., a murine IgG1, IgG2a, IgG2b, or IgG3 antibody). Of course, as the constant domains of the antibody of the invention comprise one or more mutations, the reader will understand that the isotype of such antibodies is defined by comprising first and second heavy chains that most nearly correspond with a wild-type antibody of the referenced isotype. In another particular aspect, the variable domains of part or all of the bispecific antibody, or a functional set of CDRs comprised in the FLCHCP or SLCHCP are derived from a non-human (e.g., murine) antibody, but the constant domains of the bispecific antibody are derived from a human antibody. Other types of such chimeric antibodies also are within the scope of the invention. Such humanized or otherwise chimeric bispecific antibodies can include modifications in the framework sequences necessary to ensure proper functionality, in addition to the requisite modifications with respect to a sufficient number of IPIRs.

In another particular aspect, the invention provides a method of producing a bispecific antibody comprising contacting or otherwise mixing (i) a first light chain protein (FLCP); (ii) a first heavy chain protein (FHCP) comprising the substitutions K253E, D282K, and K322D; the first light and heavy chain proteins collectively being capable of forming a FLCHCP having specificity for a first target; (iii) a second light chain protein (SLCP); and (iv) a second heavy chain protein (SHCP) comprising the substitutions K253E, D282K, and K322D; the second light and heavy chain proteins being capable of forming a SLCHCP having specificity for a second target; under conditions suitable for protein folding and association leading to the formation of a bispecific antibody, wherein either the FLCHCP or SLCHCP comprises a light chain having the substitution E15K and a heavy chain comprising the substitution K96E.

The various methods of the invention for producing the inventive BsAbs can be practiced using any suitable standard techniques. In one aspect, the production of two or more of the FLCP, FHCP, SLCP, and SHCP is accomplished by simultaneous expression of such proteins from a recombinant cell (i.e., a population of a single type of cell appropriate for producing antibodies, such as an appropriate recombinant eukaryotic or bacterial cell) encoding such proteins.

In another aspect, a BsAb of the invention can be generated by a method that comprises (a) transforming a first host cell with a first nucleic acid comprising a nucleotide sequence encoding a first polypeptide comprising the heavy chain portion of a FLCHCP; (b) transforming a second host cell with a second nucleic acid comprising a nucleotide sequence encoding a second polypeptide comprising the light chain portion of the FLCHCP; (c) transforming either (i) a third host cell with a third nucleic acid comprising third and fourth nucleic acid sequences (or third and fourth nucleic acids each respectively comprising the third and fourth nucleic acid sequences) encoding a third polypeptide comprising the light chain portion of a SLCHCP and a fourth polypeptide comprising the heavy chain portion of the SLCHCP or (iv) transforming third and fourth host cells, respectively, with such third and fourth nucleic acid molecules; (d) expressing the nucleic acid sequences; (3) purifying the expressed polypeptides; and (f) allowing the FLCP, FHCP, SLCP, SHCP generated by steps (a)-(e) to refold and associate to form the BsAb.

Thus, for example, in one exemplary aspect the invention provides a method of producing a bispecific antibody according to the invention comprising (a) expressing a first nucleic acid sequence encoding a FHCP comprising the substitutions K253E, D282K, and K322D in a first host cell, (b) expressing a second nucleic acid sequence encoding a FLCP in a second host cell, (c) expressing a third nucleic acid sequence encoding a SHCP comprising the substitutions K253E, D282K, and K322D in a third host cell, (d) expressing a fourth nucleic acid sequence encoding a SLCP in a fourth host cell, and (e) mixing the FLCP, SLCP, FHCP, and SHCP under conditions suitable for refolding and formation of a bispecific antibody therefrom so as to produce a bispecific antibody, wherein (i) the FLCP and FHCP form a FLCHCP that has specificity for a first target; (ii) the SLCP and SHCP form a SLCHCP that has specificity for a second target; and (iii) either the FLCHCP or SLCHCP comprises a light chain having the substitution E15K and a heavy chain comprising the substitution K96E.

In another exemplary example, the invention provides a method of producing a BsAb according to the invention, which comprises (a) separately expressing or co-expressing two nucleic acid sequences encoding (or otherwise generating by expression in a single cell—e.g., by cleavage of a single fusion protein comprising) a FHCP comprising the substitutions K253E, D282K, and K322D in a first host cell and a FLCP; (b) expressing a second nucleic acid sequence encoding a SHCP comprising the substitutions K253E, D282K, and K322D in a second host cell; (c) expressing a third nucleic acid sequence encoding a SLCP in a third host cell, and (d) mixing (or otherwise contacting) the FLCP, FHCP, SLCP, and SHCP under conditions suitable for refolding and the formation of tetrameric bispecific antibody therefrom, wherein (i) the FLCP and FHCP form a FLCHCP that has specificity for a first target; (ii) the SLCP and SHCP form a SLCHCP that has specificity for a second target; and (iii) either the FLCHCP or SLCHCP comprises a light chain having the substitution E15K and a heavy chain comprising the substitution K96E.

The host cells used in the above-described exemplary method or other similar methods provided by the invention are typically independently selected from eukaryotic cell and Gram-positive bacterium cells. A suitable eukaryotic cell can be selected from, for example, a mammalian cell, an insect cell, a plant cell, and a fungal cell. The host cells, can, for example, be separately selected from, e.g., the group consisting of a COS cell, a BHK cell, a HEK293 cell, a DUKX cell, a Saccharomyces spp cell, a Kluyveromyces spp cell, an Aspergillus spp cell, a Neurospora spp cell, a Fusarium spp cell, a Trichoderma spp cell, and a Lepidoptera spp cell. In separate aspects, the host cells are of the same cell type, or of different cell types (or various combinations thereof—e.g., cells 1 and 2 are of the same cell type; cells 1, 2, and 3 are of the same cell type; etc.). In one aspect, the host cells are grown in the same culture. In another aspect, some or all of the host cells are grown in separate cultures. In another aspect, the purifying step may comprise purification using an Obelix cation exchange column. In one aspect, the only antibody products expressed by the cells are those identified above (e.g., cell 1 only expresses a FHCP). In another aspect, the cells express other products, including other antibody fragments (the term “fragments” as used herein with respect to antibodies refers to a protein corresponding to a portion of a wild-type molecule or, in certain contexts, to a portion of an antibody chain, without limitation as to how such molecules are produced—i.e., antibody “fragments” need not be produced by “fragmentation” of a larger molecule, but include proteins assembled from portions of wild-type LC and/or HC proteins). In another aspect, nucleic acids are derived from one or more monoclonal antibody-producing cells. The monoclonal antibody-producing cells can, for example, be selected from a hybridoma, a polydoma, and an immortalized B-cell.

In a particular exemplary aspect, association and refolding comprises contacting (such as mixing) the polypeptides under conditions selected from: (a) a polypeptide ratio about 1:1:1:1, a temperature of about room temperature, and a pH of about 7 or (b) a polypeptide ratio of about 1:1:1:1, a temperature of about 5° C., and a pH in the range of about 8 to about 8.5. In one further aspect, the polypeptides are contacted (e.g., mixed) in a solution comprising about 0.5 M L-arginine-HCl, about 0.9 mM oxidized glutathione (GSSG), and about 2 mM EDTA. In another aspect, the ratio of the polypeptides is from about 1-2:1-2 with respect to all of the other antibodies (i.e., 1-2:1-2:1-2:1-2).

In another aspect, the production of the BsAb can alternatively or additionally (to any of the foregoing particular aspects) comprise dialyzing a solution comprising a mixture of the polypeptides.

In one aspect, the method comprises purifying a medium comprising BsAbs with an Obelix cation exchange column, and eluting purified antibodies therefrom. In a particular variation of this aspect, the method comprises at least one of the following steps: (a) applying filtrated cell culture on the column, the filtrated cell culture optionally being pH adjusted; (b) adding a solvent to the eluation buffer; and (c) eluting antibodies by increasing the salt gradient. In a particular aspect, step (c) is performed before step (b). Alternative elution strategies include, but are not limited to, the use of an elution buffer having a pH of about 6.0 and containing a salt and glycerol (e.g., about 30 mM Citrate, about 25 mM NaCl, about 30% Glycerol at a pH of about 6.0), an elution buffer having a pH of about 7.5-8.5 (e.g., Tris-buffer), a pH gradient from about pH 6.0 to a pH in the range of about 6 to about 9 (e.g., pH 7.5-8.5), and a gradient elution with salt (e.g., NaCl) from 0 to about 1M at a pH of about 6.5 to about 7.0.

The formation of the complete immunoglobulin molecule or a functional immunoglobulin fragment involves the reassembly of the heavy and light chains by disulfide bond formation which in the present invention is referred to as refolding (or refolding and association). Refolding, also termed renaturing, can be performed as described in Jin-Lian Xing et al. (2004; World J Gastroenterol 10(14):2029-2033) and Lee and Kwak (2003; Journal of Biotechnology 101:189-198). In a particular embodiment, refolding is achieved by dialysis of a mixture of heavy and light chains (or fragments thereof), the amount of heavy chains and light chains in the mixture being in the range from 1:2 to 2:1. In a further embodiment, the range is about 1:1. In the embodiment where the host cells are contained in the same culture medium, the HC and LC (or fragments thereof) self-assemble in the medium, and functional immunoglobulins or fragments can be harvested from the medium. A dialysis step of the culture media containing the mixture of HC and LC can optionally be included in the refolding process.

BsAbs also can be produced by expression of the various chains in a gram negative bacteria, such as E. coli (solely or in combination with cells of other lineage, such as eukaryotic cells). The advantages of using solely eukaryotic cells or gram positive bacterium in place of gram negative bacterium in the production of the BsAbs include—
(i) no endotoxins are present,
(ii) higher yield of protein is obtained, since there is no need for refolding protein from inclusion bodies,
(iii) full length immunoglobulins can be generated, and
(iv) the glycosylation pattern of the antibody can be modulated depending on the host organism.

Regarding item (i), endotoxins as used herein means toxic activities of enterobacterial lipopolysaccharides and are found in the outer membrane of gram-negative bacteria.

Regarding items (ii) and (v), gram negative bacteria, such as E. coli, are not well suited as production host cells if large quantities of protein are desired. The result of producing large quantities of a desired protein in E. coli is often the formation of inclusion bodies and subsequent refolding. By contrast, gram-positive bacteria have no outer membrane but a glycan layer through which proteins are secreted directly from the cytoplasm into the extracellular space. The relative simple export mechanism facilitates secretion of recombinant proteins in high yields.

Regarding item (iii), due to the large size of full length immunoglobulin molecules, these are difficult to obtain in E. coli. For a recent report on refolding complete IgG molecules produced in E. coli see Simmons et al 2002 J. Immunol. Methods 263:133-147.

Regarding item (iv), most proteins developed for pharmaceutical applications have oligosaccharides attached to their polypeptide backbone, when produced in a eukaryotic host cell. In general, sugar chains of such glycoproteins may be attached by N-glycosidic bonds to the amide group of asparagine residues or O-glycosidic bonds to the hydroxyl group of serine or threonine residues. Glycosylation is often required for proper function of the protein and ensures proper folding, function and stability. Prokaryotic organisms lack the ability to perform posttranslational modifications of proteins and glycosylation of proteins is therefore not obtained such systems. Fungi and yeast cells can be engineered to produce proteins with suitable glycosylation patterns (Ballew and Gerngross 2004 Expert Opin. Biol. Ther. 4:623-626).

The above mentioned advantages can be provided by independently producing the heavy and the light chain proteins in three or four separate host cells chosen from the group consisting of eukaryotic cells, and gram positive bacteria, as described above. In this context, the term “independently” means that the production of the respective heavy chains (HCs) and light chains (LCs) can be independently controlled or regulated by use of, e.g., different host cells, different culture media, different expression vectors, and/or different physical conditions (e.g., temperature, redox conditions, pH) of host cell culture. After production of the HC and LC chains (or fragments thereof), ex vivo refolding into a full-length antibody or antibody fragment can be achieved directly in the culture media (if the three or four separate host cells expressing the HC and LC chains, respectively, are in the same cell culture), or after one or more of joint or separate purification steps of the LCs and HCs or fragments thereof, dialysis to concentrate the HC and/or LC chain solutions and/or to change buffer, and transfer into or dilution with a particular refolding buffer.

Refolding conditions can be selected or optimized for each antibody or antibody fragment according to known methods in the art. Typically, refolding can be obtained at temperatures ranging from about +4° C. to about +40° C., or from about +4° C. to about room temperature, and at a pH ranging from about 5 to about 9, or from about 5.5 to about 8.5. Exemplary buffers that may be used for optimizing refolding include phosphate, citrate-phosphate, acetate, and Tris, as well as cell culture media with pH-regulation by CO₂ Particular refolding conditions are described in Example 1. Other exemplary refolding conditions include a HC:LC ratio of about 1:1, a temperature of about room temperature, and a neutral pH. Another exemplary refolding condition include a HC:LC ratio of about 1:1, a temperature at about 5° C., about 0.1 M Tris-HCl buffer, about 0.5 M L-arginine-HCl, about 0.9 mM oxidized glutathione (GSSG) as redox system and about 2 mM EDTA at pH of about 8.0-8.5. In one aspect, the refolding solution is dialysed against about 20 mM Tris-HCl buffer having a pH of about 7.4, and comprising about 100 mM urea until the conductivity in the equilibrated dialysis buffer has been reduced to a value in the range of about 3.0 to about 3.5 mS.

As a specific aspect of the invention, the Obelix cation exchanger can be used in the purification of antibodies. The Obelix cation exchanger binds antibodies at high conductivity and at higher pH than pI (for an antibody). This influences the purification capability. The purification can be further modulated by adding, for example, propylendiol so that a hydrophobic interaction can be utilized on this cation exchange column.

DNA encoding the monoclonal antibodies to be used in the method of the invention is readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as bacterial cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding an antibody is well known in the art (see, for example, Skerra et al., Curr. Opinion in Immunol., 5, pp. 256 (1993); and Pluckthun, Immunol. Revs., 130, pp. 151 (1992). For example, the DNA encoding an antibody chain can be isolated from the hybridoma, placed in an appropriate expression vector for transfection into an appropriate host. The host is then used for the recombinant expression of the antibody chain.

The host cell into which the DNA sequences encoding the immunoglobulin polypeptides is introduced may be any cell, which is capable of producing the posttranslational modified polypeptides if desired and includes yeast, fungi and higher eukaryotic cells. In one embodiment of the invention eukaryotic cells are selected from mammalian cells, insect cells, plant cells, and fungal cells (including yeast cells). Examples of prokaryotic cells can be Gram-negative cells such as E. coli (Cabilly et al U.S. Pat. No. 6,331,415) or Gram-positive bacteria such as Bacilli, Clostridia, Staphylococci, Lactobailli or Lactococci (de Vos et al 1997 Curr. Opin. Biotechnol. 8:547-553). Exemplary methods of expressing recombinant proteins in Gram-positive bacteria are described in U.S. Pat. No. 5,821,088. Examples of mammalian cell lines for use in the present invention are the COS-1 (ATCC CRL 1650), baby hamster kidney (BHK) and HEK293 (ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980). Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous poly-peptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (see, Gleeson et al., J. Gen. Microbiol. 132, 1986, pp. 3459-3465; U.S. Pat. No. 4,882,279). Examples of other fungal cells are cells of filamentous fungi, e.g. Aspergillus spp., Neurospora spp., Fusarium spp. or Trichoderma spp., in particular strains of A. oryzae, A. nidulans and A. niger. The use of Aspergillus spp. for the expression of proteins is described in, e.g., EP 272 277, EP 238 023, EP 184 438 The transformation of F. oxysporum may, for instance, be carried out as described by Malardier et al., 1989 (Gene 78: 147-156). The transformation of Trichoderma spp. may be performed, for instance, as described in EP 244 234.

The transformed or transfected host cell described above is then cultured in a suitable nutrient medium under conditions permitting expression of the immunoglobulin polypeptides after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The polypeptides produced by the cells may then be recovered or purified from the culture medium by conventional procedures, including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of polypeptide in question. In chromatographic procedures, the polypeptides are eluted from the column in a solution. In one aspect, the polypeptides are dialysed before or after purification from culture media to achieve polypeptides in a desired solution.

Where the FLCHCP and/or SLCHCP of a BsAb comprises variable domains of different origin from the constant domains, such as in the case portions derived from humanized antibodies, due consideration is given to the selection or screening of human variable domains, both light and heavy, to be incorporated into such humanized antibody portions, as selection of the best sequences/conditions is important to reduce antigenicity. According to the so-called “best-fit” method, a sequence of the variable domain of an antibody may be screened against a library of known human variable-domain sequences. The human sequence which is closest to that of the mouse is then accepted as the human framework (FR) for a humanized antibody (Sims et al., J. Immunol., 151, pp. 2296 (1993); Chothia and Lesk, J. Mol. Biol., 196, pp. 901 (1987)). Another method uses a particular framework from the consensus sequence of all human antibodies of a particular subgroup of light or heavy chains. The same framework can be used for several different humanized antibodies (Carter et al., Proc. Natl. Acad. Sci. U.S.A., 89, pp. 4285 (1992); Presta et al., J. Immunol., 51, pp. 1993)). Such methods can be used or adapted to the generation of BsAbs of this invention derived from, in whole or part, or comprising portions corresponding to, humanized antibodies. In other aspects, one or both portions of a BsAb can be generated from mAbs expressed from hybridomas obtained by traditional immunization methods or can correspond to portions of so-called “fully human” antibodies produced from suitable mammalian expression systems, such as the XenoMouse™ system (Abgenix—Fremont, Calif., USA) (see, e.g., Green et al. Nature Genetics 7:13-21 (1994); Mendez et al. Nature Genetics 15:146-156 (1997); Green and Jakobovits J. Exp. Med. 188:483-495 (1998); European Patent No., EP 0 463 151 B1; International Patent Application Nos. WO 94/02602, WO 96/34096; WO 98/24893, WO 99/45031, WO 99/53049, and WO 00/037504; and U.S. Pat. Nos. 5,916,771, 5,939,598, 5,985,615, 5,998,209, 5,994,619, 6,075,181, 6,091,001, 6,114,598 and 6,130,364)).

Bispecific antibodies of the invention can be specific for any suitable pair of first and second targets.

In one aspect, the invention provides BsAbs wherein the first or second target is an immune cell regulatory molecule (such as, e.g., CD4/CD8, CD28, CD26, CTLA-4, ICOS, or CD11a), such as a co-stimulatory molecule (e.g., CD28), or a regulatory receptor (e.g., CTLA-4) (typically where that portion of the BsAb is derived from a CTLA-4 inhibitory antibody), and the second target is an appropriate lymphocyte activating receptor. Other suitable first or second targets associated with immune cells include T cell-associated molecules, such as TCR/CD3 or CD2; NK cell-associated targets such as FcγRIIIa (CD16), CD38, CD44, CD56, or CD69; granuloctye-associated targets such as FcγRI (CD64), FcαRI (CD89), and CR3 (CD11b/CD18); monocyte/macrophage-associated targets (such as FcγRI (CD64), FcoRI (CD89), CD3 (CD11b/CD18), or mannose receptor; dendritic cell-associated targets such as FcγRI (CD64) or mannose receptor; and erythrocyte-associated targets such as CR I (CD35). Examples of target combinations previously or currently in clinical development include CD3×EGP-2; CD3×folate receptor; CD3×CD19; CD16×CD30; CD16×HER-2/neu; CD64×HER-2/neu; and CD64×EGF receptor (see, e.g., an Spriel et al., Immunology Today, 21(8):391-397 (2000)). Various other suitable combinations of targets are described in Kontermann et al. (2005) supra, and include, e.g., EpCAM, BCL-1, FAP, OKT9, CD40, CEA, IL-6, CD19, CD20, MUC-1, EGFR, Pgp, Lys, C1q, DOTA, and EDG.

Known cancer antigens, which may be targeted by the FLCHCP and/or SLCHCP of the BsAb include, without limitation, c-erbB-2 (erbB-2; which also is known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and α-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kD found in adenocarcinoma of colon, as well as cancers of the lung and genitourinary tract. Yet another class of cancer antigens is those antigens unique to a particular tumor, referred to sometimes as “tumor specific antigens,” such as heat shock proteins (e.g., hsp70 or hsp90 proteins) from a particular type of tumor. Other targets include the MICA/B ligands of NKG2D. These molecules are expressed on many types of tumors, but not normally on healthy cells.

Additional specific examples of cancer antigens that may be targeted by the FLCHCP and/or SLCHCP include epithelial cell adhesion molecule (Ep-CAM/TACSTD1), mucin 1 (MUC1), carcinoembryonic antigen (CEA), tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA), RAGE (renal antigen), α-fetoprotein, CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

The FLCHCP and/or SLCHCP can generally target protein antigens, carbohydrate antigens, or glycosylated proteins. For example, a BsAb can target glycosylation groups of antigens that are preferentially produced by transformed (neoplastic or cancerous) cells, infected cells, and the like (cells associated with other immune system-related disorders). In one aspect, the antigen is a tumor-associated antigen. In an exemplary aspect, the antigen is MUC1. In another particular aspect, the antigen is one of the Thomsen-Friedenreich (TF) antigens (TFAs).

Antibodies to a number of these and other cancer antigens are known and additional antibodies against these or other cancer antigens can readily be prepared by an ordinarily skilled artisan using routine experimentation. For example, antibodies to CEA have been developed as described in UK 2 276 169, wherein the variable sequences of such antibodies also is provided. Other examples of known anti-cancer antigen antibodies include anti-oncofetal protein mAbs (see U.S. Pat. No. 5,688,505), anti-PSMA mAbs (see, e.g., U.S. Pat. No. 6,649,163), and anti-TAG-72 antibodies (see U.S. Pat. No. 6,207,815). Anti-CD19 Antibodies include anti-B4 (Goulet et al. Blood 90: 2364-75 (1997)), B43 and B43 single-chain Fv (FVS191; Li et al., Cancer Immunol. Immunother. 47:121-130 (1998)). Antibodies have been reported which bind to phosphatidyl-serine and not other phospholipids (e.g., Yron et al., Clin. Exp. 1 mmol. 97: 187-92) (1994)). A dimeric single-chain Fv antibody construct of monoclonal CC49 recognizes the TAG-72 epitope (Pavlinkova et al., Clin. Cancer Res. 5: 2613-9 (1999)). Additional anti-TAG-72 antibodies include B72.3 (Divgi et al., Nucl. Med. Biol. 21: 9-15 (1994)) and those disclosed in U.S. Pat. No. 5,976,531. Anti-CD38 antibodies are described in, e.g., Ellis et al., J. Immunol. 155: 925-37 (1995) (mAb AT13/5); Flavell et al., Hematol. Oncol. 13: 185-200 (1995) (OKT10-Sap); and Goldmacher et al., 84: 3017-25 (1994)). Anti-HM1.24 antibodies also are known (see, e.g., Ono et al., Mol. Immuno. 36: 387-95 (1999)). Cancer antigen-binding sequences can be obtained from these antibodies or cancer antigen-binding variants thereof can be generated by standard techniques to provide suitable VH and VL (or corresponding CDR) sequences. See also, Stauss et al.: TUMOR ANTIGENS RECOGNIZED BY T CELLS AND ANTIBODIES and Taylor and Frances (2003) and Durrant et al., Expert Opin. Emerging Drugs 8(2):489-500 (2003) for a description of additional tumor specific antigens which may be targeted by BsAbs of the invention.

BsAbs of the invention also can exhibit specificity for a non-cancer antigen cancer-associated protein. Such proteins can include any protein associated with cancer progression. Examples of such proteins include angiogenesis factors associated with tumor growth, such as vascular endothelial growth factors (VEGFs), fibroblast growth factors (FGFs), tissue factor (TF), epidermal growth factors (EGFs), and receptors thereof; factors associated with tumor invasiveness; and other receptors associated with cancer progression (e.g., one of the HER1-HER4 receptors).

Antibodies against these and other cancer-associated proteins are known or can be readily developed by standard techniques. Well-known antibodies against advantageous targets include anti-CD20 mAbs (such as Rituximab and HuMax-CD20), anti-Her2 mAbs (e.g., Trastuzumab), anti-CD52 mAbs (e.g., Alemtuzumab and Campath® 1H), anti-EGFR mAbs (e.g., Cetuximab, HuMax-EGFr, and ABX-EGF), Zamyl, Pertuzumab, anti-A33 antibodies (see U.S. Pat. No. 6,652,853), anti-aminophospholipid antibodies (see U.S. Pat. No. 6,406,693), anti-neurotrophin antibodies (U.S. Pat. No. 6,548,062), anti-C3b(i) antibodies (see U.S. Pat. No. 6,572,856), anti-MN antibodies (see, e.g., U.S. Pat. No. 6,051,226), anti-mts1 mAbs (see, e.g., U.S. Pat. No. 6,638,504), and anti-VEGF mAbs (e.g., bevacizumab), edrecolomab, tositumomab, lbritumomab tiuxetan, and gemtuzumab ozogamicin. Sequences can be obtained from these or similar antibodies and/or variants derived therefrom for incorporation to a BsAb of the invention.

BsAbs of the invention alternatively can be specific for a virus-associated target, such as an HIV protein (e.g., gp120 or gp41). Antibodies against GP120 are known that can be used for generation of such BsAbs (see, e.g., Haslin et al., Curr Opin Biotechnol. 2002 December; 13(6):6214 and Chaplin, Med. Hypotheses. 1999 February; 52(2):13346). Antibodies against other HIV proteins have been developed that can be useful in the context of generating such BsAbs (see, e.g., Re et al., New Microbiol. 2001 April; 24(2):197-205; Rezacova et al. J Mol Recognit. 2002 September-October; 15(5):272-6; Stiegler et al., Journal of Antimicrobial Chemotherapy (2003) 51, 757-759; and Ferrantelli et al., Curr Opin Immunol. 2002 August; 14(4):495-502). Antibodies against other suitable viral targets, such as CMV, also are known (see, e.g., Nokta et al., Antiviral Res. 1994 May; 24(1):17-26). Targeting of other viruses, such as hepatitis C virus (HCV) also may be advantageous.

Antibodies can be readily generated against such targets and such antibodies or already available antibodies can be characterized by routine methods so as to determine VH and VL sequences (or more particularly VH and VL CDRs), which can be “inserted” (incorporated, e.g., by genetic engineering) into the FLCHCP and SLCHCP of the bispecific antibody of the invention.

The structure of variable domains for a number of antibodies against such targets already are publicly available. For example, the sequences presented in Table 2, represent exemplary VH and VL sequences for an anti-CD16 antibody, which may be incorporated in a BsAb of the invention:

TABLE 2
Exemplary anti-CD16 VH and VL Sequences

| | | | |
|---|---|---|---|
| VH | SEQ ID | murine | MDRLTSSFLLLIVPAYVLSQVTLKESGPGILQPS |
| | NO:1 | | QTLSLTCSFSGFSLRTSGMGVGWIRQPSGKGLEW |
| | | | LAHIWWDDDKRYNPALKSRLTISKDTSSNQVFLK |
| | | | IASVDTADTATYYCAQINPAWFAYWGQGTLVTVS |
| | | | A |
| VL | SEQ ID | murine | METDTILLWVLLLWVPGSTGDTVLTQSPASLAVS |
| | NO:2 | | LGQRATISCKASQSVDFDGDSFMNWYQQKPGQPP |
| | | | KLLIYTTSNLESGIPARFSASGSGTDFTLNIHPV |
| | | | EEEDTATYYCQQSNEDPYTFGGGTKLEIK |

Anti-CD20 antibodies, from which anti-CD20 FLCHCP or SLCHCP sequences can be obtained or derived are well known. For example, the US FDA approved anti-CD20 antibody, RITUXIMAB™ (IDEC C2B8; RITUXAN; ATCC No. HB 11388), has been used regularly to treat humans for cancer. Ibritumomab, is the murine counterpart to RITUXIMAB™ (Wiseman et al., Clin. Cancer Res. 5: 3281s-6s (1999)). Other reported anti-CD20 antibodies include the anti-human CD20 mAb 1F5 (Shan et al., J. Immunol 162:6589-95 (1999)), the single chain Fv anti-CD20 mouse mAb 1H4 (Haisma et al., Blood 92: 184-90 (1998)) and anti-B1 antibody (Liu et al., J. Clin. Oncol. 16: 3270-8 (1998)). In the instance of 1H4, a fusion protein was created reportedly fusing 1H4 with the human β-glucuronidase for activation of the prodrug N-[4-doxorubicin-N-carbonyl(-oxymethyl)phenyl] O-β-glucuronyl carbamate to doxorubicin at the tumor cite (Haisma et al. 1998). Rituximab and related anti-CD20 antibodies are further described in International Patent Application WO 94/11026 and Liu et al., J. Immunol. 139(10):3521-3526 (1987). Other anti-CD20 antibodies are described in, e.g., International Patent Application WO 88/04936. Exemplary anti-CD20 VH and VL sequences are provided in Table 3:

TABLE 3
Exemplary anti-CD20 VH and VL Ab Sequences

| | | |
|---|---|---|
| Ab | VH | VL |

| | | |
|---|---|---|
| 1 | MDFQVQIISFLLISASVIMSRGQIVLSQSPAILSA | MGWSLILLFLVAVATRVLSQVQLQQPGAELVK |
| | SPGEKVTMTCRASSSVSYIHWFQQKPGSSPK | AGASVKMSCKASGYTFTSYNMHWVKQTPGR |
| | PWIYATSNLASGVPVRFSGSGSGTSYSLTISR | GLEWIGAIYPGNGDTSYNQKFKGKATLTADKS |
| | VEAEDAATYYCQQWTSNPPTFGGGTKLEIK | SSTAYMQLSSLTSEDSAVYYCARSTYYGGDW |
| | | YFNVWGAGTVVTVSA |
| 2 | MAQVQLRQPGAELVKPGASVKMSCKASGYTF | MAQIVLSQSPAILSASPGEKVTMTCRASSSLSF |
| | TSYNMHWVKQTPGQGLEWIGAIYPGNGDTSY | MHWYQQKPGSSPKPWIYATSNLASGVPARFS |
| | NQKFKGKATLTADKSSSTAYMQLSSLTSEDSA | GSGSGTSYSLTISRVEAEDAATYFCHQWSSN |
| | VYYCARSHYGSNYVDYFDYWGQGTLVTVSTG | PLTFGAGTKVEIKRK |
| 3 | QVQLQESGPSLVKPGASVKIVCKASGYTFTRL | ADGVPSRFSGSGSGTQFSLKINRLQPEDFGN |
| | | YYCQHFWSTPWTFGGGTKLEIKRA |
| 4 | QVQLVQSGAELVKPGASVKMSCKASGYTFTS | DIVLSQSPAILSASPGEKVTMTCRASSSVSYM |
| | YNMHWVKQTPGQGLEWIGAIYPGNGDTSYNQ | HWYQQKPGSSPKPWIYATSNLASGVPARFSG |
| | KFKGKATLTADKSSSTAYMQLSSLTSEDSAVY | SGSGTSYSLTISRVEAEDAATYYCQQWISNPP |
| | YCARAQLRPNYWYFDVWGAGTTVTVS | TFGAGTKLELK |

SEQ ID NOS:3-9, respectively (left-to-right, line-to-line).

In another aspect, a BsAb of the invention may target tissue factor (TF). Therapeutic use of mouse mAbs against TF is described in, e.g., U.S. Pat. Nos. 6,001,978 and 5,223,427. International Application No. WO 99/51743 describes human/mouse chimeric monoclonal antibodies directed against human TF. European patent application No. 833911 relates to CDR-grafted antibodies against human TF. Presta L. et al., Thrombosis and Haemostasis, Vol. 85 (3) pp. 379-389 (2001) relates to humanized antibody against TF. Human TF antibodies are further described in, e.g., International Patent Applications WO 03/029295 and WO 04/039842; WO 89/12463 and U.S. Pat. No. 6,274,142 (Genentech); WO 88/07543, U.S. Pat. No. 5,110,730, U.S. Pat. No. 5,622,931, U.S. Pat. No. 5,223,427, and U.S. Pat. No. 6,001,978 (Scripps); and WO 01/70984 and U.S. Pat. No. 6,703,494 (Genentech). Table 4 lists a set of exemplary anti-TF CDRs which may be (with suitable framework sequences) incorporated into a FLCHCP or SLCHCP of a BsAb of the invention:

TABLE 4
Exemplary anti-Tissue Factor Antibody CDRs

| | | |
|---|---|---|
| CDR-H1 | SEQ ID NO:10 | GFNIKEYYMH |
| CDR-H2 | SEQ ID NO:11 | LIDPEQGNTIYDPKFQD |
| CDR-H3 | SEQ ID NO:12 | DTAAYFDY |
| CDR-L1 | SEQ ID NO:13 | RASRDIKSYLN |
| CDR-L2 | SEQ ID NO:14 | YATSLAE |
| CDR-L3 | SEQ ID NO:15 | LQHGESPWT |

As described above, BsAbs of the invention that are specific for Her-2/neu may be advantageous (e.g., in the treatment of cancer). Several antibodies have been developed against Her-2/neu, including trastuzumab (e.g., HERCEPTIN™—see, e.g., Fornier et al., Oncology (Huntingt) 13: 647-58 (1999)), TAB-250 (Rosenblum et al., Clin. Cancer Res. 5: 865-74 (1999)), BACH-250 (Id.), TA1 (Maier et al., Cancer Res. 51: 5361-9 (1991)), and the monoclonal antibodies (mAbs) described in U.S. Pat. Nos. 5,772,997; 5,770,195 (mAb 4D5; ATCC CRL 10463); and 5,677,171. Conjugated anti-Her-2 antibodies also are known (see, e.g., Skrepnik et al., Clin. Cancer Res. 2: 1851-7 (1996) and U.S. Pat. No. 5,855,866). Anti-Her-2 antibodies and uses thereof are further described in, e.g., U.S. Pat. No. 6,652,852 and International Patent Applications WO 01/00238, WO 01/00245, WO 02/087619, and WO 04/035607. Exemplary anti-Her2 VH and VL sequences that may be incorporated into a FLCHCP or SLCHCP of a BsAb of the invention are set forth in Table 5:

TABLE 5
Exemplary anti-Her-2 VH and VL Sequences

| | |
|---|---|
| VH | EVQLVESGGGLVQPGGSLRLSCAASGFNIKDTYIHWVRQAPGKGL |
| | EWVARIYPTNGYTRYADSVKGRFTISADTSKNTAYLQMNSLRAEDT |
| | AVYYCSRWGGDGFYAMDYWGQGTLVTVSSASTKGPSVFPLAPSSKS |
| | TSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHT |
| | (SEQ ID NO:17) |
| VH | EVQVQQSGPEVVKTGASVKISCKASGYSFTGYFINWVKKNSGKSPE |
| | WIGHISSSYATSTYNQKFKNKAAFTVDTSSSTAFMQLNSLTSEDSA |
| | VYYCVRSGNYEEYAMDYWGQGTSVTVSS |
| | (SEQ ID NO:18) |
| VH | QIQLVQSGPELKKPGETVKISCKASGYTFTNYGMNWVKQAPGESLK |
| | WMGWLNTNTGEPTYAEDFKGRFAFSLGTSASTAYLRINNVKDEDTA |
| | TYFCARWGRDDVGYWGQGTTLIVSS |
| | (SEQ ID NO:19) |
| VH | EVQLVESGGGLVQPKGSLKL |
| | (SEQ ID NO:20) |
| VL | DIQMTQSPSSLSASVGDRVTITCRASQDVNTAVAWYQQKPGKAPKL |
| | LIYSASFLYSGVPSRFSGSRSGTDFTLTISSLQPEDFATYYCQQHY |
| | TTPPTFGQGTKVEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSFNRGEC |
| | (SEQ ID NO:21) |
| VL | DVLMTQTPLSLPVSLGDQASISCRSGQSIVHSNGNTYLEWYLQKPG |
| | QSPKLLIYRVSNRFSGVPDRFSGSGSGSDFTLKISRVEAEDLGVYY |
| | CFQYSHVPWTFGGGTKLEIKR |
| | (SEQ ID NO:22) |
| VL | DIVLTQTPSSLPVSVGEKVTMTCKSSQTLLYSNNQKNYLAWYQQKP |
| | GQSPKLLISWAFTRKSGVPDRFTGSGSGTDFTLTIGSVKAEDLAVY |
| | YCQQYSNYPWTFGGGTRLEIKR |
| | (SEQ ID NO:23) |
| VL | DIVMTQSQKFMSTSVVDRIS |
| | (SEQ ID NO:24) |

In another exemplary aspect, the invention provides BsAbs that are specific for an epidermal growth factor (EGF) receptor (EGFR or EGF-R). Epidermal growth factor-receptor (EGF-R) binds to EGF, a mitogenic peptide. Anti-EGF-R antibodies and methods of preparing them are known (see, e.g., U.S. Pat. Nos. 5,844,093 and 5,558,864 and European Patent No. 706,799A). The US FDA approved the anti-EGFR mAb ERBITUX™ (Cetuximab) for the treatment of certain cancers in February 2004. Erbitux slows cancer growth by targeting EGFR. Exemplary anti-EGF-R VH and VL sequences are set forth in Table 6:

TABLE 6
Exemplary anti-EGER VH and VL Sequences

| | |
|---|---|
| VH | VL |

| | |
|---|---|
| QVQLQESGPELVRPGASVKMSCKAS | DIELTQSPASLAASVGETVTITCRASENIYYSLA |
| GYTFTTYWIHWMKQRPGQGLQWIG | WYQQKQGKSPQLLIYSASALEDGVPSRFSGS |
| MIDPSNSETRLNQNFRDKATLSVDKS | GSGTQYSLKINNMQPEDTATYFCKQTYDVPW |
| SNKAYMQLSSLTSEDSAIYYCARWDY | TFGGGTKLEIKRA |
| GSGHFDYWGQGTTVTVSS | |
| QVQLQESGPELVKPGALVKISCKASG | DIELTQSPASLAVSLGQRATISCRASESVDNFG |
| YTFTSYWMHWVKQRPGQGLEWIGEI | ISFMNWFQQKPGQPPKLLIYGASNQGSGVPA |
| DPSDSYTNYNQKFKGKATLTVDKSS | RFSGSGSGTDFSLNIHPLEEDDTAMYFCQQSK |
| NTAYMQLSSLTSEDSAVYYCARSDY | EVPLTFGAGTKLEIKR |
| GSSHFDYWGQGTTVTVSS | |
| EVQLQQSGAELVKPGASVKLSCKAS | DIELTQSPASLAVSLGQRATISCRASESVDNFG |
| GYTFTSYWMHWVKQRPGQGLEWIG | ISFMNWFQQKPGQPPKLLIYGASNQGSGVPA |
| EIDPSDSYTNYNQKFKGKATLTVDKS | RFSGSGSGTDFSLNIHPLEEDDTAMYFCQQSK |
| SSTAYMQLSSLTSEDSAVYYCARSDY | EVPLTFGAGTKLELKRA |
| GSSHFDYWGQGTTVTVSS | |
| EVKLQQSGPELVKPGASVKMSCKAS | DIELTQSPTTMAASPGEKITITCSASSSISSNYL |
| GYAFISFVMHWVKQKPGQGLEWIGFI | HWYQQKPGFSPKLLIYRTSNLASGVPARFSGS |
| NPYNDGTKYNEKFKDKATLTSDKSSS | GSGTSYSLTIGTMEAEDVATYYCQQGSSIPRT |
| TAYMELSSLTSEDSAVYYCASGDYD | FGGGTKLEIKR |
| RAMDYWGQGTTVTVSS | |

SEQ ID NOS:25-31, respectively (left-to-right, row-by-row).

In another aspect, the invention provides BsAbs that are specific for a VEGF receptor (VEGFR or VEGF-R), such as a KDR receptor.

Numerous types of antibodies against VEGFRs are known. The anti-VEGFR mAb AVASTIN™ (Bevacizumab), for example, was approved by the US FDA for the treatment of cancer in humans in February 2004.

Exemplary anti-VEGFR CDR sequences are set forth in Table 7:

TABLE 7
Exemplary Anti-VEGR CDR Sequences

| | | | | | | |
|---|---|---|---|---|---|---|
| Ab | CDR L1 | CDR L2 | CDR L3 | CDR H1 | CDR H2 | CDR H3 |

| | | | | | | |
|---|---|---|---|---|---|---|
| 1 | RASQSVSS | DSSNRAT | LQHNTFPPT | GFTFSSYSMN | SISSSSSYIYYA | VTDAFDI |
| | YLA | | | | DSVKG | |
| 2 | RASQGISS | AASSLQT | QQANRFPPT | GFTFSSYSMN | SISSSSSYIYYA | VTDAFDI |
| | RLA | | | | DSVKG | |
| 3 | AGTTTDLT | DGNKRPS | NSYVSSRFYV | GFTFSSYSMN | SISSSSSYIYYA | VTDAFDI |
| | YYDLVS | | | | DSVKG | |
| 4 | SGSTSNIG | NNNQRPS | AAWDDSLNGHWV | GGTFSSYAIS | GGIIPIFGTANYA | GYDYYDSSGVA |
| | TNTAN | | | | QKFQG | SPFDY |

SEQ ID NOS:32-55, respectively (left-to-right, row-by-row).

In a further aspect, the invention provides BsAbs that are specific for CD52 (CAMPATH-1). CD52 is a 21-28 kD cell surface glycoprotein expressed on the surface of normal and malignant B and T lymphocytes, NK cells, monocytes, macrophages, and tissues of the male reproductive system (see, e.g., Hale, Cytotherapy. 2001; 3(3):13743; Hale, J Biol Regul Homeost Agents. 2001 October-December; 15(4):386-91; Domagala et al., Med Sci Monit. 2001 March-April; 7(2):325-31; and U.S. Pat. No. 5,494,999).

CD52 antibodies are well known in the art (see, e.g., Crowe et al., Clin. Exp. Immunol. 87 (1), 105-110 (1992); Pangalis et al., Med Oncol. 2001; 18(2):99-107; and U.S. Pat. No. 6,569,430). Alemtuzumab (Campath®) is an FDA approved anti-CD52 antibody which has been used in the treatment of chronic lymphocytic leukemia.

Exemplary anti-CD52 VH and VL sequences are set forth in Table 8:

TABLE 8
Exemplary Anti-CD52 VL and VH Sequences

| | | |
|---|---|---|
| Set | VL | VH |

| | | |
|---|---|---|
| 1 | DIKMTQSPSFLSASVGDRVTLNCK | EVKLLESGGGLVPGGSMRLSCAGSGF |
| | ASQNIDKYLNWYQQKLGESPKLLI | TFTDFYMNWIRQPAGKAPEWLGFIRDK |
| | YNTNNLQTGIPSRFSGSGSGTDFT | AKGYTTEYNPSVKGRFTISRDNTQNML |
| | LTISSLQPEDVATYFCLQHISRPRT | YLQMNTLRAEDTATYYCAREGHTAAPF |
| | FGTGTKLELKR | DYWGQGVMVTVSS |
| | (SEQ ID NO:56) | (SEQ ID NO:57) |
| 2 | DIQMTQSPSSLSASVGDRVTITCK | QSVQLQESGPGLVRPSQTLSLTCTVSG |
| | ASQNIDKYLNWYQQKPGKAPKLLI | STFSDFYMNWVRQPPGRGLEWIGFIR |
| | YNTNNLQTGVPSRFSGSGSGTDF | DKAKGYTTEYNPSVKGRVTMLVDTSKN |
| | TFTISSLQPEDIATYYCLQHISRPRT | QFSLRLSSVTAADTAVYYCAREGHTAA |
| | FGQGTKVEIKR | PFDYWGQGSLVTVSS |
| | (SEQ ID NO:58) | (SEQ ID NO:59) |

In another illustrative aspect, the invention provides BsAbs that specifically bind to CD33. CD33 is a glycoprotein expressed on early myeloid progenitor and myeloid leukemic (e.g., acute myelogenous leukemia, AML) cells, but not on stem cells. IgG₁ monoclonal antibodies against CD33 have been prepared in mice (M195) and in humanized form (HuM195) (see, e.g., Kossman et al., Clin. Cancer Res. 5: 2748-55 (1999)). MYLOTARG™ (gemtuzumab ozogamicin a conjugate derived from an anti-CD33 mAb (conjugated to the bacterial toxin calicheamicin), for example, has been approved by the US FDA since 2000 for use in the treatment of CD33 positive acute myeloid leukemia (see, e.g., Sievers et al., Blood Cells Mol Dis. 2003 July-August;31(1):7-10; Voutsadakis, et al., Anticancer Drugs. 2002 August; 13(7):685-92; Sievers et al., Curr Opin Oncol. 2001 November; 13(6):522-7; and Co et al., J. Immunol. 148 (4), 1149-1154 (1992)).

An exemplary anti-CD33 light chain sequence is
(SEQ ID NO:60)
MNKAMRBPMEKDTLLLWVLLLWVPGSTGDIVLTQSPASLAVSLGQRATISCRASESVDNYGI
SFMNWFQQKPGQPPKLLIYAASNQGSGVPARFSGSGSGTDFSLNIHPMEEDDTAMYFCQQ
SKEVPWTFGGGTKLEIK.
An exemplary anti-CD33 heavy chain sequence is
(SEQ ID NO:61)
MGWSWIFLFLLSGTAGVHSEVQLQQSGPELVKPGASVKISCKASGYTFTDYNMHWVKQSH
GKSLEWIGYIYPYNGGTGYNQKFKSKATLTVDNSSSTAYMDVRSLTSEDSAVYYCARGRPA
MDYWGQGTSVTVSS.

In a further aspect, the invention provides BsAbs that specifically bind MUC-1. MUC-1 is a carcinoma associated mucin. MUC-1 antibodies are known and demonstrated to possess anti-cancer biological activities (see, e.g., Van H of et al., Cancer Res. 56: 5179-85 regarding e.g., mAb hCTMO1). For example, the anti-MUC-1 monoclonal antibody, Mc5, has reportedly suppressed tumor growth (Peterson et al., Cancer Res. 57: 1103-8 (1997)).

Sequences
(SEQ ID NO:62)
DIVVTQESALTTSPGETVTLTCRSSTGAVTTSNYANWVQEKPDHLFTGLI
GGTNNRAPGVPARFSGSLIGDKAALTITGAQTEDEAIYFCALWYSNHWVF
GGGTKLTVLGSE
and
(SEQ ID NO:63)
QVQLQESGGGLVQPGGSMKLSCVASGFTFSNYWMNWVRQSPEKGLEWVAE
IRLKSNNYATHYAESVKGRFTISRDDSKSSVYLQMNNLRAEDTGIYYCTG
VGFAYWGQGTTVTVS,
represent, respectively, anti-MUC-1 VL and VH
sequences.

In yet another illustrative aspect, the invention provides BsAbs that specifically bind to CD22. CD22 is a cell surface antigen expressed on normal human B cells and some neoplastic B cells. Several monoclonal anti-CD22 antibodies have been created, including HD6, RFB4, UV22-2, Tol5, 4 KB128, a humanized anti-CD22 antibody (hLL2), and a bispecific F(ab′)₂ antibody linked to saporin (see, e.g., Li et al. Cell. Immunol. 111: 85-99 (1989); Mason et al., Blood 69: 836-40 (1987); Behr et al., Clin. Cancer Res. 5: 3304s-14s (1999); and Bonardi et al., Cancer Res. 53: 3015-21 (1993)).

Exemplary anti-CD22 VH and VL sequences are set forth in Table 9:

TABLE 9
Exemplary Anti-CD22 VH and VL Sequences

| | |
|---|---|
| VH | VL |

| | |
|---|---|
| EVQLVQSGAEVKKPGASVKVSCKASGYRFTNY | DVVVTQSPSSLSASVGDRVTITCRSSQSLAN |
| WIHWVRQAPGQGLEWIGGINPGNNYATYRRKF | SYGNTFLSWYLHKPGKAPQLLIYGISNRFSGV |
| QGRVTMTADTSTSTVYMELSSLRSEDTAVYYC | PDRFSGSGSGTDFTLTISSLQPEDFATYYCLQ |
| TREGYGNYGAWFAYWGQGTLVTVSS | GTHQPYTFGQGTKVEIKR |
| (SEQ ID NO:64) | (SEQ ID NO:65) |
| EVQLVQSGAEVKKPGASVKVSCKASGYRFTNY | DVQVTQSPSSLSASVGDRVTITCRSSQSLAN |
| WIHWVRQAPGQGLEWIGGINPGNNYATYRRNL | SYGNTFLSWYLHKPGKAPQLLIYGISNRFSGV |
| KGRVTMTADTSTSTVYMELSSLRSEDTAVYYC | PDRFSGSGSGTDFTLTISSLQPEDFATYYCLQ |
| TREGYGNYGAWFAYWGQGTLVTVSS | GTHQPYTFGQGTKVEIKR |
| (SEQ ID NO:66) | (SEQ ID NO:67) |
| EVQLVQSGAEVKKPGASVKVSCKASGYRFTNY | DVVVTQTPLSLPVSFGDQVSISCRSSQSLAN |
| WIHWVRQAPGQGLEWIGGINPGNNYATYRRNL | SYGNTFLSWYLHKPGQSPQLLIYGISNRFSG |
| KGRATLTADTSTSTVYMELSSLRSEDTAVYYCT | VPDRFTGSGSGTDFTLKISTIKPEDLGMYYCL |
| REGYGNYGAWFAYWGQGTLVTVSS | QGTHQPYTFGGGTKLEIKR |
| (SEQ ID NO:68) | (SEQ ID NO:69) |
| EVQLQQSGTVLARPGASVKMSCKASGYRFTN | DVVVTQTPLSLPVSFGDQVSISCRSSQSLAN |
| YWIHWVKQRPGQGLEWIGGINPGNNYTTYKRN | SYGNTFLSWYLHKPGQSPQLLIYGISNRFSG |
| LKGKATLTAVTSASTAYMDLSSLTSEDSAVYYC | VPDRFTGSGSGTDFTLKISTIKPEDLGMYYCL |
| TREGYGNYGAWFAYWGQGTLVTVSS | QGTHQPYTFGGGTKLEIKR |
| (SEQ ID NO:70) | (SEQ ID NO:71) |

In still another illustrative aspect, the invention provides BsAbs that specifically bind to CD4. CD4 is a transmembrane glycoprotein of the immunoglobulin superfamily, expressed on developing thymocytes, major histocompatibility class II (class II MHC)-restricted mature T lymphocytes and, in humans, on cells of the macrophage/monocyte lineage. On lymphoid cells, CD4 plays a critical role during thymocyte ontogeny and in the function of mature T cells. CD4 binds to non-polymorphic regions of class II MHC acting as a co-receptor for the T-cell antigen receptor (TCR). It increases avidity between thymocytes and antigen-presenting cells and contributes directly to signal transduction through association with the Src-like protein tyrosine kinase p56lck. CD4 is also a co-receptor for the human and simian immunodeficiency viruses (HIV-1, HIV-2, and SIV). Specifically, CD4 is a receptor for human immunodeficiency virus (HIV)-gp120 glycoprotein. Clinically, CD4 antibodies may be used to achieve immunological tolerance to grafts and transplants; treat autoimmune diseases and immune deficiency-related disorders such as, e.g., lupus, diabetes, rheumatoid arthritis, etc.; treat leukemias and lymphomas expressing CD4; as well as to treat HIV infection. Bowers et al., Int J Biochem Cell Biol. 1997 June; 29(6):871-5 (see also Olive and Mawas, Crit Rev Ther Drug Carrier Syst. 1993; 10(1):29-63; Morrison et al., J Neurosci Res. 1994 May 1; 38(1):1-5); Lifson et al., Immunol Rev. 1989 June; 109:93-117.

Exemplary anti-CD4 VH and VL sequences are, respectively,

(SEQ ID NO:72)
DIQMTQSPASLSASVGETVTFTCRASENIYSYLAWYQQKQGKSPQLLVHDAKTLAEGVPSR
FSGGGSGTQFSLKINTLQPEDFGTYYCQHHYGNPPTFGGGTKLEIK
and

(SEQ ID NO:73)

QVQLKQSGPGLVQPSQSLSITCTVSGFSLTTFGVHWVRQSPGKGLEWLGVIWRSGITDYNV
PFMSRLSITKDNSKSQVFFKLNSLQPDDTAIYYCAKNDPGTGFAYWGQGTLVTVSA.

### EXPERIMENTAL METHODS AND DATA

The following exemplary experimental methods and data are presented to better illustrate various aspects of the invention, and related illustrative enabling technology, but in no event should be viewed as limiting the scope of the invention.

### Example 1

### Identification of Amino Acid Residues Responsible for Ionic Interactions in Immunoglobulins

References to heavy chain constant region position numbers here specifically indicate the position of the wild-type constant region sequence starting from the beginning (N-terminus) of CH1 (according to UNIPROT-id:IGHG1_HUMAN). For constant light chain positions, numbering is according to Uniprot-id:KAC_HUMAN. The amino acids responsible for the ionic interactions in human IgG1s were identified using an analysis of X-ray structures available for the CH3-CH3 domain-domain interactions of both the GM and KM allotypes, and X-ray structures available for CH1-CKappa and CH1-CLambda interactions.

Specifically, the following KM X-ray structures were analysed: 1 HZH, 1ZA6, 10QX, 10QO, 1L6X; the following GM X-ray structures were analysed: 1T89, 1T83, 1IIX, 1H3X; the following CH1-Ckappa X-ray structures were analysed: 1TZG, 1HZH; and the following CH1-Clambda X-ray structure was analysed: 2RCS.

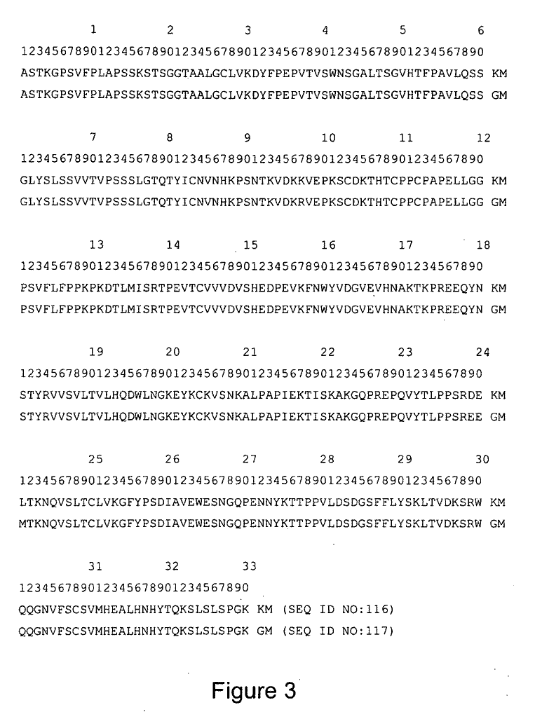

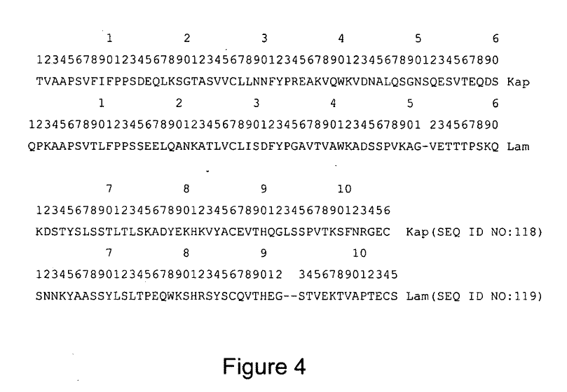

The constant part of the heavy chain IgG1 sequence comes in 2 allotypes: KM and GM. The constant part of the light chain can come from 2 loci: Kappa and Lambda. When analyzing the relevant 3D-PDB structures, combinations of KM/GM and Kappa/Lambda appear. An analysis of the differences between KM and GM sequences is shown in FIG. 3. An analysis of the sequence differences between Kappa and Lambda sequences are shown in FIG. 4.

For the KM/GM sequence comparison, only the following differences were observed: K97R, D239E, L241M. This finding is relevant in that, e.g., one of the ionic interactions involves D239.

For Kappa/Lambda sequences there are several differences and different lengths. This means that the positions of the ionic interactions are different in Kappa and Lambda due to different lengths, but not due to a different mechanism.

Using standard methods in available molecular modelling packages, e.g., MOE (Molecular Operating Environment) software available from Chemical Computing Group (www.chemcomp.com), intramolecular ionic interactions were identified. This analysis specifically led to the to the identification of 6 CH3-CH3 GM ionic interactions, 6 CH3-CH3 KM ionic interactions, 2 CH1-CKappa and 2 CH1-CLambda interactions all listed below

CH3-CH3 KM:
D239-K322
E240-K253
D282-K292

CH3-CH3 GM:
E239-K322
E240-K253
D282-K292

CKappa-CH1:
E15-K96
D14-K101

CLambda-CH1
E16-K96
E17-K30

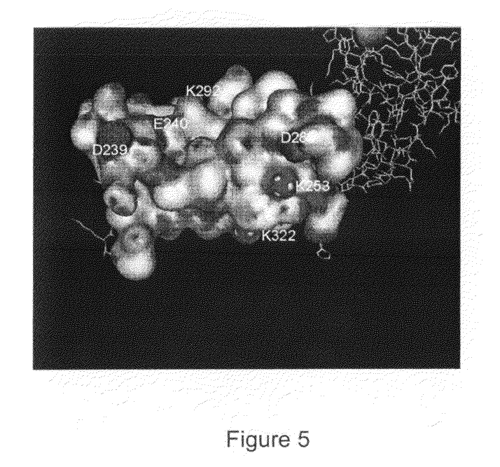

FIG. 5 is a molecular surface illustration, showing the interaction points of one CH3 surface, generated using the data identified by this analysis.

### Example 2

### Modification of Amino Acids in First and Second LCHCPs to Promote Heterodimer (BsAb) Formation

As briefly described already, amino acid residues involved in the above-described interactions were subjected to substitutions in two LCHCPs (from different antibodies having different specificities) in order to increase the energy of (required for) homodimeric interactions and thereby favor heterodimeric interactions (and thus, formation of a BsAb). The same principle can be applied for heavy-light chain interactions.

Examples:

CH3-Unmodified<->CH3-Unmodified
D239<->K322
E240<->K253
K292<->D282
K322<->D239
K253<->E240
D282<->K292

Suggesting the modifications K322D, K253E, D282K in chain A and D239K, E240K, K292D in chain B leads to a CH3-Modified-A<->CH3-Modified-B interaction with only matching pairs
D239<->K322
E240<->K253
K292<->D282
D322<->K239
E253<->K240
K282<->D292

Whereas the CH3-Modified-A<->CH3-Modified-A interaction becomes:
D239<->D322
E240<->E253
K292<->K282
D322<->D239
E253<->E240
K282<->K292

With only charge repulsion pairs (i.e., pairs of residues that would not form ionic interactions such as those that occur normally in a human IgG at these positions).

A similar approach can be applied for the GM, and Heavy light-chain interactions.

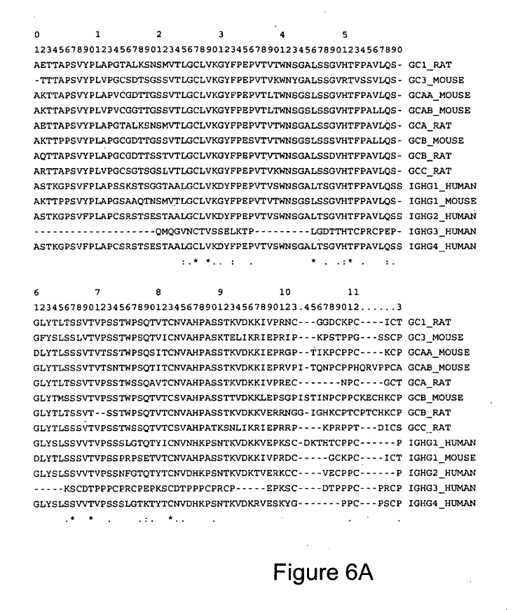

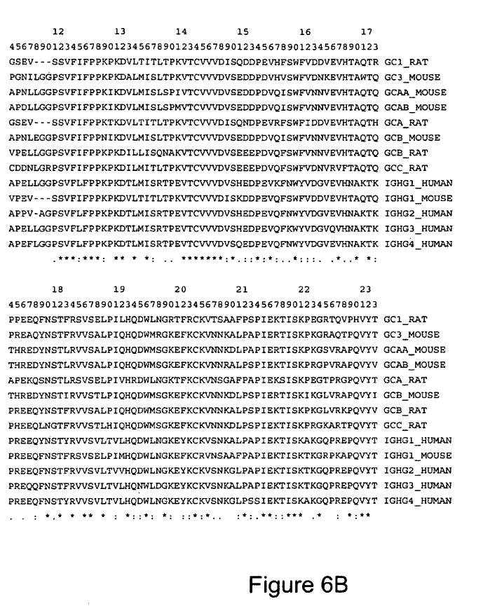

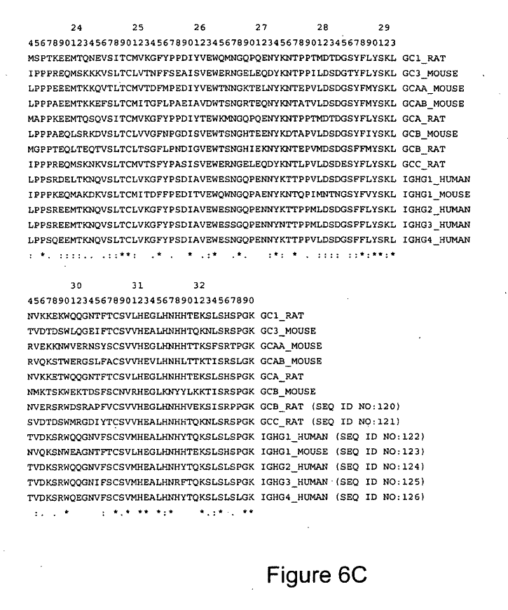

Based on the high homology of immunoglobulins, a structural homology can be predicted, the interactions described above have counterparts for other human isotypes (IgG2-4), as well as, e.g., mouse and rat IgGs. To identify the corresponding residues, an alignment has been performed and is shown in FIG. 6.

### Conservation of Heavy Chain:

D239 or E239 is conserved in all subtypes and species

K322 is conserved in all subtypes and species

E240 is conserved in humans, rat igg1, igg2a, mouse igg2a

K253 is conserved in humans, rat igg1, igg2a

D282 is conserved in all subtypes and species except for mouse igg1

K322 is conserved in all subtypes and species

K96 is conserved in all subtypes and species except for human igg3

K101 or R101 is conserved in all subtypes and species except for mouse igg2b

K30 is conserved in all subtypes and species except for human igg3

### Conservation of Light Chain:

E15 is conserved in human and mice (rat not investigated)

D14 not conserved

E16 is conserved in human and mice (rat not investigated)

E17 is conserved in human and mice (rat not investigated)

This analysis demonstrates that methods of the invention (e.g., involving modification of amino acid residues involved in ionic interactions so as to promote formulation of bispecific antibody molecules of interest) can be readily applied to antibody sequences derived from a variety of species and subtypes. Nearly all residues involved in ionic interactions in human IgG molecules, for example, are conserved in all subtypes and species, meaning that modification of residues at most of the positions identified in respect of human IgG molecules in such other antibody amino acid sequences will lead to similar results in terms of practicing the methods described herein and that only a minimal amount of routine work is necessary to identify a full complement of ionic interaction pairs in immunoglobulin species derived from other organisms or antibody subtypes (it is noted that D14 is not critical for dimerization of the heavy chains).

### Example 3

### Recombinant Cloning of Two Human Antibodies Recognizing Independent Targets

An anti-human tissue factor antibody, HuTF33-F9, that immunoreacts with human tissue factor (TF) to inhibit the binding of coagulation factor VIIa (FVIIa) (described in US20050106139-A1) (herein frequently labeled “TF”) and antibody HuKIR1-7F9 that binds Killer Immunoglobulin-like Inhibitory Receptors (“KIRs”) KIR2DL1, KIR2DL2, and KIR2DL3 (described in WO2006003179-A2) (herein frequently abbreviated KIR), were used to prepare the bispecific anti-TF/anti-KIR antibodies described here. The anti-TF antibody is a fully human IgG1 antibody and the anti-KIR antibody is a fully human IgG4 antibody.

Isolation of total RNA from hybridoma cells: 4×10⁶ hybridoma cells (HuTF-33F9) and (HuKIR1-7F9) secreting antibodies against two independent antigens were used for isolation of total RNA using RNeasy Mini Kit from Qiagen. The cells were pelleted for 5 min at 1000 rpm and disrupted by addition of 350 μl RLT buffer containing 10 μl/ml β-mercaptoethanol. The lysate was transferred onto a QIAshredder column from Qiagen and centrifuged for 2 min at maximum speed. The flow through was mixed with 1 volume 70% ethanol. Up to 700 μl sample was applied per RNeasy spin column and centrifuged at 14000 rpm and the flow through discarded. 700 μl RW1 buffer was applied per column and centrifuged at 14000 rpm for 15 s to wash the column. The column was washed twice with 500 μl RPE buffer and centrifuged for 14000 rpm for 15 s. To dry the column, it was centrifuged for additionally 2 min at 14000 rpm. The column was transferred to a new collection tube and the RNA was eluted with 50 μl of nuclease-free water and centrifuged for 1 min at 14000 rpm. The RNA concentration was measured by absorbance at OD=260 nm. The RNA was stored at −80° C. until needed.
cDNA synthesis: 1 μg RNA was used for first-strand cDNA synthesis using SMART RACE cDNA Amplification Kit from Clontech. For preparation of 5′-RACE-Ready cDNA, a reaction mixture containing RNA isolated, as described above, back primer 5′-CDS primer back, and SMART II A oligo, was prepared and incubated at 72° C. for about 2 min., and subsequently cooled on ice for about 2 min. before adding 1× First-Strand buffer, DTT (20 mM), dNTP (10 mM) and PowerScript Reverse Transcriptase. The reaction mixture was incubated at 42° C. for 1.5 hour and Tricine-EDTA buffer was added and incubated at 72° C. for 7 min.
Amplification and cloning of human light (VLCL) and human IgG1 AND IgG4 heavy chains (VHCH1-3 IgG1 and VHCH1-3 IgG4): A PCR (Polymerase Chain Reaction) reaction mixture containing 1× Advantage HF 2 PCR buffer, dNTP (10 mM) and 1× Advantage HF 2 polymerase mix was established for separate amplification of both VLCL, VHCH1-3 IgG1, and VHCH1-3 IgG4 from cDNA made as above.
For amplification of VHCH1-3 IgG1 and VHCH1-3 IgG4 the following primers were used:

| UPM (Universal Primer Mix): | |
|---|---|
| 5′-CTAATACGACTCACTATAGGGCAAGCAGTGGTATCAACGCAGAGT-3′ | (SEQ ID NO:74) |
| and | |
| 5′-CTAATACGACTCACTATAGGG-3′ | (SEQ ID NO:75) |
| HuIgG1 (for amplification of VHCH1-3 IgG1): | |
| 5′-TCATTTACCCGGGGACAGGGAG-3′ | (SEQ ID NO:76) |
| HuIgG4 (for amplification of VHCH1-3 IgG4): | |
| 5′-TCATTTACCCAGAGACAGGGAGA-3′ | (SEQ ID NO:77) |
| For amplification of VLCL the following primers were used: | |
| UPM (Universal Primer Mix): | |
| 5′-CTAATACGACTCACTATAGGGCAAGCAGTGGTATCAACGCAGAGT-3′ | (SEQ ID NO:78) |
| 5′-CTAATACGACTCACTATAGGG-3′ | (SEQ ID NO:79) |
| HuKLC: | |
| 5′-CTAACACTCTCCCCTGTTGAAGCTC-3′ | (SEQ ID NO:80) |

Three rounds of PCR were conducted as follows. Round 1: PCR is run for 5 cycles at 94° C. for 5 s and 72° C. for 3 min. Round 2: PCR is run for 5 cycles at 94° C. for 5 s, 70° C. for 10 s, and 72° C. for 1 min. Round 3: PCR is run for 28 cycles at 94° C. for 5 s, 68° C. for 10 s, and 72° C. for 1 min.
The PCR products were analyzed by electrophoresis on a 1% agarose gel and the DNA purified from the gel using QIAEX11 agarose gel extraction kit from Qiagen. The purified PCR products were introduced into PCR4-TOPO vector using TOPO TA Cloning kit from Invitrogen and used for transformation of TOP10 competent cells. A suitable amount of colonies were analyzed by colony PCR using Taq polymerase, 1× Taq polymerase buffer, dNTP (10 mM) and the following primers and PCR program:

| | |
|---|---|
| M13forward: | |
| 5′-GTAAAACGACGGCCAG-3′ | (SEQ ID NO:81) |
| M13reverse: | |
| 5′-CAGGAAACAGCTATGAC-3′ | (SEQ ID NO:82) |

PCR Program: 25 cycles are run at 94° C. for 30 s, 55° C. for 30 s, and 72° C. for 1 min.

Plasmid DNA from clones comprising VLCL, VHCH1-3 IgG1 and VHCH1-3 IgG4 inserts, respectively, was extracted and sequenced using primer M13forward and M13reverse listed above.

### Example 4

### Construction and Expression of Antibody Variants

Mutations were introduced in the constant regions of both IgG1 and IgG4 heavy chains using Multi-Site Directed Mutagenesis (Stratagene cat. No. 200514) and the cloned VLCL, VHCH1-3 IgG1, and VHCH1-3 IgG4 as templates and the oligonucleotides presented in table 10:

TABLE 10

| | | | | Construct of | |
|---|---|---|---|---|---|
| | | | SEQ. | mutated | Construct of variable + |
| Subtype | Mutation | Oligo | ID | constant part | mutated constant part |
| IgG1 heavy | K253E | KK216 | 82 | HC1-IgG1 | TF-HC1-IgG1 |
| chain | D282K | KK218 | 83 | | |
| | K322D | KK218a | 84 | | |
| | K96E | KK221 | 85 | HC2-IgG1 | KIR-HC2-IgG1 |
| | D239K | KK223 | 86 | | |
| | E240K | KK223 | 86 | | |
| | K292D | KK225 | 87 | | |
| IgG4 heavy | K253E | KK352 | 89 | HC1-IgG4 | TF-HC1-IgG4 |
| chain | D282K | KK353 | 90 | | |
| | K322D | KK354 | 91 | | |
| | K96E | KK355 | 93 | HC2-IgG4 | KIR-HC2-IgG4 |
| | D239K | KK356 | 94 | | |
| | E240K | KK356 | 94 | | |
| | K292D | KK357 | 95 | | |
| Kappa light | — | | | LC1 | TF-LC1 |
| chain | | | | | |
| | E14K | KK228 | 88 | LC2 | KIR-LC2 |

KK216: 5′-GCCTGGTCGAGGGCTTCTATCC-3′ (SEQ ID NO: 83)
KK218: 5′-CCTCCCGTGCTGAAATCCGACG-3′ (SEQ ID NO: 84)
KK218a: 5′-CCACTACACGCAGGACAGCCTCTCCCTGTCCCC-3′ (SEQ ID NO: 85)
KK221: 5′-CCCAGCAACACCAAGGTGGACGAGAGAGTTGA-3′ (SEQ ID NO: 86)
KK223: 5′-TGCCCCCATCCCGGAAGAAAATGACCAAG-3′ (SEQ ID NO: 87)
KK225: 5′-TCCTTCTTCCTCTATAGCGATCTCACCGTGG-3′ (SEQ ID NO: 88)
KK228: 5′-CATCTTCCCGCCATCTGATAAGCAGTTGAA-3′ (SEQ ID NO: 89)
KK352: 5′-GCCTGGTCGAAGGCTTCTACCCCAG-3′ (SEQ ID NO: 90)
KK353: 5′-CTCCCGTGCTGAAATCCGACGGCTC-3′ (SEQ ID NO: 91)
KK354: 5′-ACTACACACAGGACAGCCTCTCCC-3′ (SEQ ID NO: 92)
KK220: 5′-TCAACTCTCTCGTCCACCTTGG-3′ (SEQ ID NO: 93)
KK355: 5′-CAAGGTGGACGAGAGAGTTGAGTCC-3′ (SEQ ID NO: 94)
KK356: 5′-CCCATCCCAGAAGAAGATGACCAAG-3′ (SEQ ID NO: 95)
KK357: 5′-CTCTACAGCGATCTAACCGTGGACA-3′ (SEQ ID NO: 96)

Introduction of Constant Domain Variants into Mammalian Expression Vectors:

The mutated constant regions were each introduced into mammalian expression vectors suitable for transient expression in HEK293 6E cells in the following manner. The constant heavy chain regions were amplified with primers (Table 11)) designed to introduce a NheI site in the 5′ end and a BamHI site in the 3′ end. The PCR product was digested with NheI and BamHI prior to ligation into the NheI/BamHI site of pJSV002. The constant light chain regions were amplified with primers containing a 5′ BsiWI site and a 3′ XbaI site, respectively, and introduced into the BslWI/XbaI site of pJSV001.

TABLE 11

Oligonucleotides used for amplification of mutated constant
chains of human IgG1 and IgG4

| | | |
|---|---|---|
| Ab1H-IgG1-for: | 5′-GCTAGCACCAAGGGCCCATCCGTC-3′ | (SEQ ID NO: 97) |
| Ab1H-IgG1-back: | 5′-GCGCAGATCTTCATTTACCCGGGGACAGGGAGAGGCTGTCCT-3′ | (SEQ ID NO: 98) |
| Ab1L-IgG1-for: | 5′-CGGCCGTACGGTGGCTGCACCATCTGTCTTC-3′ | (SEQ ID NO: 99) |
| Ab1L-IgG1-back: | 5′-GCGCTCTAGACTAACACTCATTCCTGTTGAAGCT-3′ | (SEQ ID NO: 100) |
| Ab2H-IgG1-for: | 5′-GCTAGCACCAAGGGCCCATCCGTC-3′ | (SEQ ID NO: 97) |
| Ab2H-IgG1-back: | 5′-GCGCAGATCTTCATTTACCCGGGGACAGGGAG-3′ | (SEQ ID NO: 101) |
| Ab2L-IgG1-for: | 5′-CGGCCGTACGGTGGCTGCACCATCTGTCTTC-3′ | (SEQ ID NO: 99) |
| Ab2L-IgGl-back: | 5′-GCGCTCTAGACTAACACTCATTCCTGTTGAAGCT-3′ | (SEQ ID NO: 100) |
| Ab1H-IgG4-for: | 5′-GCTAGCACCAAGGGCCCATCCGTC-3′ | (SEQ ID NO: 97) |
| Ab1H-IgG4-back: | 5′-GAAGATCTTCATTTACCCAGAGACAGGGAGAGGCTGTCCT-3′ | (SEQ ID NO: 102) |
| Ab1L-IgG4-for: | 5′-CGGCCGTACGGTGGCTGCACCATCTGTCTTC-3′ | (SEQ ID NO: 99) |
| Ab1L-IgG4-back: | 5′-GCGCTCTAGACTAACACTCATTCCTGTTGAAGCT-3′ | (SEQ ID NO: 100) |
| Ab2H-IgG4-for: | 5′-GCTAGCACCAAGGGCCCATCCGTC-3′ | (SEQ ID NO: 97) |
| Ab2H-IgG4-back: | 5′-GAAGATCTTCATTTACCCAGAGACAGGGAGAG-3′ | (SEQ ID NO: 103) |
| Ab2L-IgG4-for: | 5′-CGGCCGTACGGTGGCTGCACCATCTGTCTTC-3′ | (SEQ ID NO: 99) |
| Ab2L-IgG4-back: | 5′-GCGCTCTAGACTAACACTCATTCCTGTTGAAGCT-3′ | (SEQ ID NO: 100) |

Introduction of Variable Antibody Genes into Mammalian Expression Vectors:

Based on the sequence data, primers were designed for the amplification of the variable light (VL) and variable heavy (VH) chain genes, of HuTF-33F9 and HuKIR1-7F9, respectively (Table 12).

TABLE 12
Oligonucleotides used for amplification of antibody variable regions

| | | |
|---|---|---|
| HuTF-33F9-VL-for: | 5′-GCGCAAGCTTGCCACCATGGAAGCCCCAGCTCAGCTTC-3′ | (SEQ ID NO: 104) |
| HuTF-33F9-VL-back: | 5′-GCGCCGTACGTTTGATCTCCACCTTGGTCCCT-3′ | (SEQ ID NO: 105) |
| HuTF-33F9-VH-for: | 5′-GGCCGCGGCCGCACCATGGAGTTTGGGCTGAG-3′ | (SEQ ID NO: 106) |
| HuTF-33F9-VH-back: | 5′-GCCGGCTAGCTGAGGAGACGGTGACCAG-3′ | (SEQ ID NO: 107) |
| HuKIR1-7F9-VL-for: | 5′-GCGCAAGCTTGCCACCATGGAAGCCCCAGCTCAGCTTC-3′ | (SEQ ID NO: 108) |
| HuKIR1-7F9-VL-back: | 5′-GCGCCGTACGTTTGATCTCCAGCTTGGTCC-3′ | (SEQ ID NO: 109) |
| HuKIR1-7F9-VH-for: | 5′-GCGGCCGCCATGGACTGGACCTGGAGGTTC-3′ | (SEQ ID NO: 110) |
| HuKIR1-7F9-VH-back: | 5′-GCCGGCTAGCTGAGGAGACGGTGACCGTGGT-3′ | (SEQ ID NO: 111) |

The variable regions were formatted by PCR to include a Kozak sequence, leader sequence, and unique restriction enzyme sites. For the VL, this was achieved by designing 5′ PCR primers to introduce a HindIII site, the Kozak sequence, and to be homologous to the 5′ end of the leader sequence of the variable light chain region. The 3′ primer was homologous to the 3′ end of the variable region and introduced a BsiWI site at the 3′ boundary of the variable region. The VH region was generated in a similar fashion except that a NotI and a NheI site were introduced in the 5′ and 3′ end instead of HindIII and BsiWI, respectively.

The amplified gene products were each cloned into their own eukaryotic expression vectors using standard techniques and leading to the constructs presented in Table 10.

### VH Deletion for BsIg Ratio Determination:

In order to show that the mutations in the constant region has an effect on the assembly of the antibody heavy chains and to quantify the amount of bispecific immunoglobulin (“BsIg”) formed, a construct was made which only comprised the constant domain of antibody 1. The constant region of antibody 1 (IgG1) was amplified with KK391: 5′-GCGGCCGCCATGGCTAGCACCAAGGGCCCATC-3′ (SEQ ID NO: 112) containing a NotI site and a start codon in the 5′-end, and KK226: 5′-GCGCAGATCTTCATTTACCCGGGGACAGGGAG-3′ (SEQ ID NO: 113) containing a stop codon and a BglII site in the 3′-end. The PCR product was digested with NotI and BglII, respectively, and introduced into the NotI/BamHI site of pJSV002.

Due to the difference in protein size between the truncated version and the intact heavy chain it will be possible to determine if the mutations push the reaction towards assembly of BsIg by analyzing the transiently expressed polypeptides using an Agilent 2100 Bioanalyzer (Agilent Technologies) and the protocol provided by the manufacturer.

### S-S-Bridge Deletion:

In order to show that ionic interactions are sufficient for assembly/dimerization of the Fc domain the Cysteine residues in the IgG hinge region was substituted with Alanine residues. The Cys residues were substituted with Alanine residues in the TF-H1-IgG1, KIR-H2-IgG1, TF-H1-IgG4 and KIR-H2-IgG4 constructs by site directed mutagenesis (Stratagene cat. No. 200514) using the oligonucleotides IgG1-Cys-Ala:

| | |
|---|---|
| 5′-CTCACACAGCGCCACCGGCGCCAGCACCTGAAC-3′ | (SEQ ID NO: 114) |
| on DNA from the TF-H1-IgG1 and KIR-H2-IgG1 constructs, and | |
| IgG4-Cys-Ala: | |
| 5′-GGTCCCCCAGCGCCATCAGCGCCAGCACCTGAG-3′ | (SEQ ID NO: 115) |
| on DNA from the TF-H1-IgG4 and KIR-HC-IgG4 constructs, | |
| respectively. | |

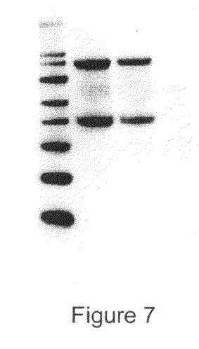

Dimerization of first and second antibody Fc domains was observed, indicating (i) factors other than disulphide bridge formation are sufficient for heterodimerization of antibodies and (ii) that the introduced mutations in the Fc domains of antibody 1 and 2 do not abolish the ability of the two chains to form intact antibodies (FIG. 7).

### Expression of Bispecific Constructs:

The cloned DNAs described above are introduced into HEK293 6E cells using Lipofectamine™ 2000 (Cat. No. 11668-019, Invitrogen) and grown for 6 days according to the manufacturer's recommendations before supernatants were analyzed.

### Example 5

### Analysis of Antibody Variants

### SDS-PAGE and Western Blot Analysis:

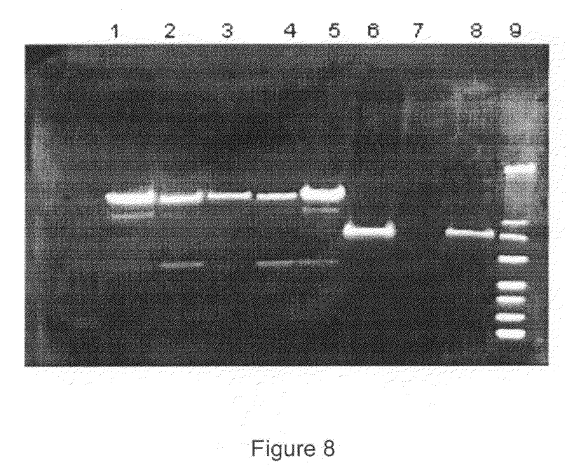

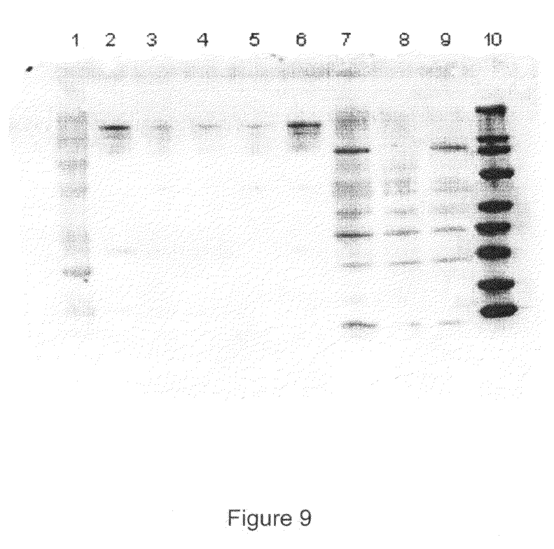

The supernatant from the transfected HEK293 6E cells described above were analyzed by SDS-PAGE using Novex 4-12% Bis-Tris and Tris Acetate 4-8% gels. Anti-human IgG1 and anti-human IgG kappa light chain antibodies were used for detection in Western blot analysis. The results in FIGS. 8 and 9 demonstrate that the introduced mutations do not disrupt the ability of the antibody polypeptide chains to dimerize.

### Surface Plasmon Resonance:

A Biacore 3000 optical biosensor was used to evaluate the affinities of the expressed antibodies towards human TF and human KIR2DL3. In order to determine affinities, approximately 10000 RU (RU=Resonance Units) of antigen was immobilized to the sensor surface by EDC/NHS coupling chemistry. Thereafter, the antibody was injected into the flow cell with a flow rate of about 5 μl/min for about 3 min. and allowed to associate with its respective antigen (human TF or human KIR2□L3). Following the association phase, the surface was washed with running buffer (HBS-EP, pH 7.4, containing 0.005% detergent P20) at a flow rate of 5 μl/min for 2 min. The sensorgram data were analyzed using the Bia evaluation software 3.0.

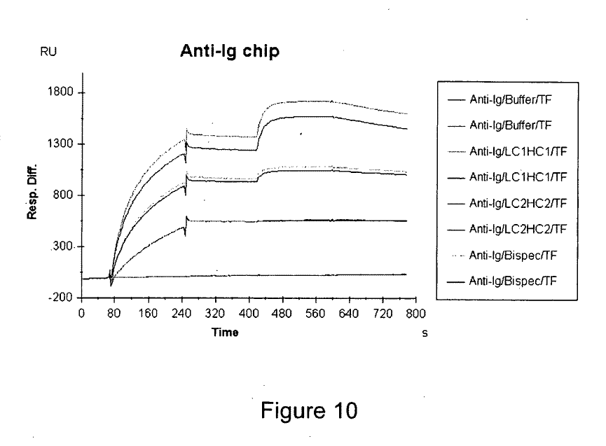

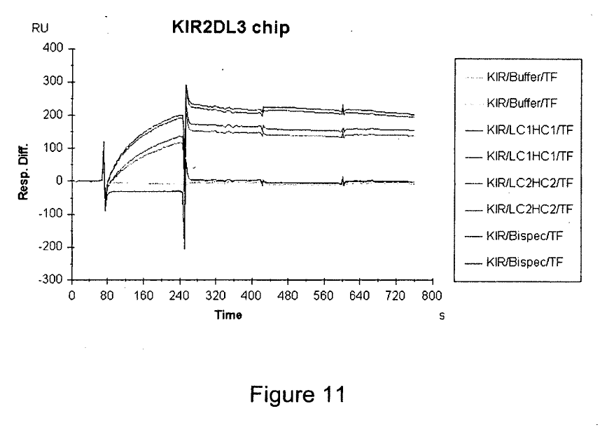

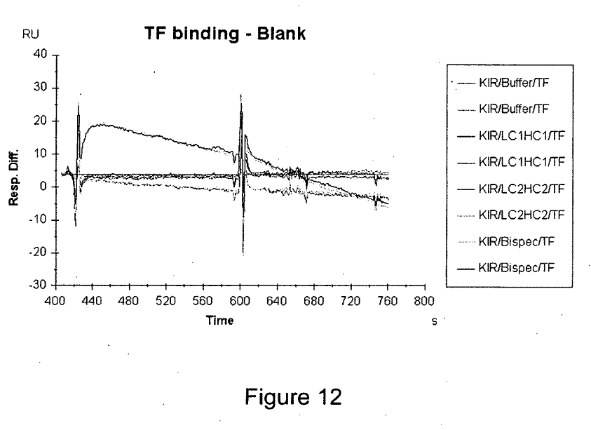

The results demonstrate the presence of bispecific antibodies which are also recognized by IgG specific antibody (FIGS. 10-12). In FIG. 12, binding to TF was observed, indicating formation of bispecific antibodies.

The same type of experiment was made with IgG4 HC. Results similar to those obtain for IgG1 HC were obtained in the Western blot-analysis, while no conclusive results could be obtained from initial Biacore analysis due to, e.g., high back-ground binding.

### Quantification of Properly Assembled BsIg:

Using an Agilent 2100 Bioanalyzer, it will be possible to compare and quantify the ratio of BsIg with unwanted antibody contaminants.

### Example 6

### Bispecific Immunoglobulin Ratio Determination

In order to show that the mutations in the constant regions had an effect on the assembly of the antibody heavy chains and to quantify the amount of bispecific immunoglobulin (“BsIg”) formed, constructs were made which only comprised the hinge region and Fc part of Ab1 and Ab2 (both IgG1 and IgG4), respectively. Due to the difference in protein size between the truncated version and the intact heavy chain, the effect of the mutations on pushing the reaction towards assembly of BsIg was assayed by analyzing the transiently expressed polypeptides by SDS-PAGE and by using an Agilant 2100 Bioanalyzer (Agilent Technologies) and the protocol provided by the manufacturer.

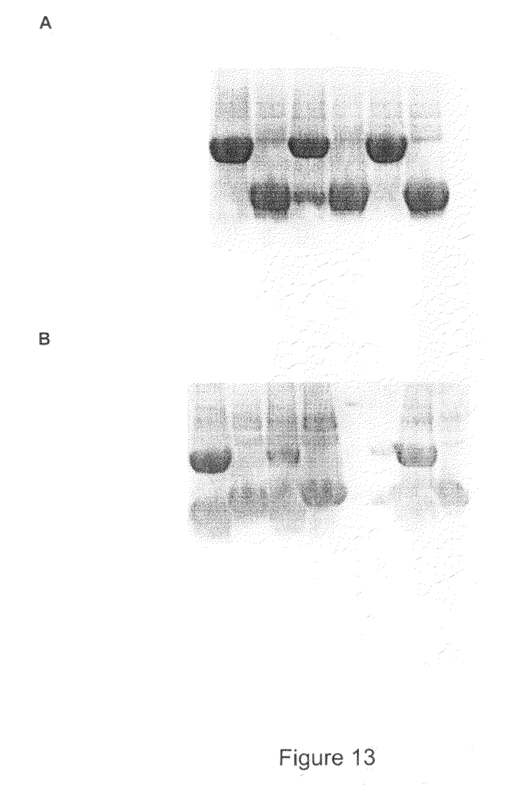

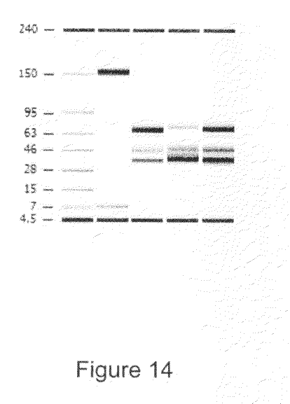

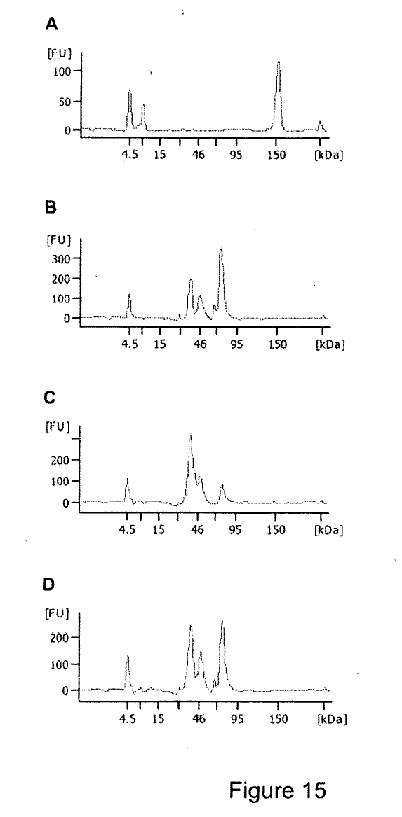

FIGS. 13 to 15 show that dimerization of Ab2 heavy chain (in both IgG1 and IgG4 formats) is reduced as a result of the mutations introduced into the human IgG1 and IgG4 Fc domains, respectively.

### EXEMPLARY EMBODIMENTS

The following are exemplary embodiments of the present invention:

1. A bispecific antibody comprising (a) a first light-heavy chain pair (“FLCHCP”) having specificity for a first target, the first heavy chain comprising the substitutions K253E, D282K, and K322D; and (b) a second light-heavy chain pair (“SLCHCP”) having specificity for a second target, the second heavy chain comprising the substitutions D239K, E240K, and K292D; wherein either the FLCHCP or SLCHCP comprises a light chain having the substitution E15K and a heavy chain comprising the substitution K96E.

2. The antibody of embodiment 1, wherein the FLCHCP, SLCHCP, or both comprise human antibody CDRs.

3. The antibody of embodiment 1, wherein the FLCHP, SLCHCP, or both comprise murine antibody CDRs.

4. The antibody of any one of embodiments 1-3, wherein the FLCHP, SLCHCP, or both comprise CDRs derived from a species that is different from the species that the constant domain of the antibody is derived from.

5. The antibody of any one of embodiments 14, wherein the antibody has a human IgG4 isotype.

6. The antibody of any one of embodiments 1-4, wherein the antibody has a human IgG1 isotype.

7. The antibody of any one of embodiments 1-4, wherein the antibody has a murine IgG1 isotype.

8. The antibody of any one of embodiments 1-7, wherein the antibody comprises at least a portion of an IgG Fc domain which increases the in vivo half-life of the antibody.

9. The antibody of any one of embodiments 1-8, wherein the antibody comprises a functional IgG Fc domain.

10. The antibody of any one of embodiments 1-7, wherein the antibody lacks a functional IgG Fc domain or comprises a non-functional IgG Fc domain.

11. The antibody of any one of embodiments 1-10, wherein the FLCHCP and SLCHCP comprise different light chains.

12. The antibody of any one of embodiments 1-11, wherein the antibody is free of (a) non-naturally occurring intramolecular cysteine-cysteine disulfide bonds; (b) protuberance and cavity modifications in the multimerization domain; (c) artificial hydrophilic or hydrophobic sequence modifications comprising two or more contiguous amino acid residue substitutions; or (d) any combination of (a)-(c).

13. The antibody of any one of embodiments 1-12, wherein the antibody is free of any linkage to one or more additional antibody molecules or fragments by covalent linkage.

14. A method of producing a bispecific antibody comprising contacting

(i) a first light chain protein (“FLCP”);

(ii) a first heavy chain protein (“FHCP”) comprising the substitutions K253E, D282K, and K322D;

wherein the FLCP and FHCP are capable of forming a FLCHCP having specificity for a first target;

(iii) a second light chain protein (“SLCP”); and

(iv) a second heavy chain protein (“SHCP”) comprising the substitutions K253E, D282K, and K322D;

wherein the SLCP and SHCP are capable of forming a SLCHCP having specificity for a second target,

under conditions suitable for the formation of a bispecific antibody comprising the FLCHCP and SLCHCP, wherein either the FLCHCP or SLCHCP comprises a light chain having the substitution E15K and a heavy chain comprising the substitution K96E.

15. The method of embodiment 14, wherein the FLCP, FHCP, SLCP, and SHCP are expressed in a single cell.

16. The method of embodiment 14, wherein the FLCP and FHCP are expressed in a first cell, the SLCP is expressed in a second cell, and the SHCP is expressed in a third cell.

17. The method of embodiment 14, wherein the FLCP, FHCP, SLCP, and SHCP are all expressed in different cells.

18. The method of any one of embodiments 14-17, wherein the cell(s) used to produce the FLCP, FHCP, SLCP, and SHCP are selected from eukaryotic and bacterial cells.

19. A method of producing a bispecific antibody comprising:

(a) identifying pairs of amino acid residues involved in intramolecular ionic interactions in a wild-type tetrameric antibody molecule of the isotype in an organism,

(b) preparing (i) FLCP and FHCP capable of forming a FLCHCP comprising at least some substitutions of amino acid residues involved in such wild-type antibody intramolecular interactions and having specificity for a first target and (ii) SLCP and SHCP capable of forming a SLCHCP having specificity for a second target and comprising an amino acid sequence complementary to the first light chain-heavy chain pair in terms of such intramolecular ionic interactions, the FLCHCP and SLCHCP collectively comprising substitution of a sufficient number of amino acid residues involved in such wild-type antibody intramolecular interactions that bispecific tetramers comprising the FLCHCP and SLCHCP form more frequently than molecules comprising only the FLCHCP or SLCHCP when the FLCP, FHCP, SLCP, and SHCP are permitted to mix, and

(c) mixing the FLCP, FHCP, SLCP, and SHCP or the FLCHCP and SLCHCP under suitable conditions so as to produce a bispecific antibody.

20. A bispecific antibody comprising a FLCHCP having specificity for a first target and a sufficient number of substitutions in its heavy chain constant domain with respect to a corresponding wild-type antibody of the same isotype to significantly reduce the formation of first heavy chain-first heavy chain dimers and a SLCHCP comprising a heavy chain having a sequence that is complementary to the sequence of the FLCHCP heavy chain sequence with respect to the formation of intramolecular ionic interactions, wherein the FLCHCP or the SLCHCP comprises a substitution in the light chain and complementary substitution in the heavy chain that reduces the ability of the light chain to interact with the heavy chain of the other LCHCP.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

1. A bispecific antibody comprising (a) a first light-heavy chain pair (“FLCHCP”) having specificity for a first target, the first heavy chain comprising the substitutions K253E, D282K, and K322D; and (b) a second light-heavy chain pair (“SLCHCP”) having specificity for a second target, the second heavy chain comprising the substitutions D239K, E240K, and K292D; wherein either the FLCHCP or SLCHCP comprises a light chain having the substitution E15K and a heavy chain comprising the substitution K96E.

2. The antibody of claim 1, wherein the FLCHCP, SLCHCP, or both comprise human antibody CDRs.

3. The antibody of claim 1, wherein the FLCHP, SLCHCP, or both comprise murine antibody CDRs.

4. The antibody of claim 1, wherein the FLCHCP, SLCHCP, or both comprise CDRs derived from a species that is different from the species that the constant domain of the antibody is derived from.

5. The antibody of claim 1, wherein the antibody has a human IgG4 isotype.

6. The antibody of claim 1, wherein the antibody has a human IgG1 isotype.

7. The antibody of claim 1, wherein the antibody comprises at least a portion of an IgG Fc domain which increases the in vivo half-life of the antibody.

8. The antibody of claim 1, wherein the antibody comprises a functional IgG Fc domain.

9. The antibody of claim 1, wherein the antibody lacks a functional IgG Fc domain or comprises a non-functional IgG Fc domain.

10. The antibody of claim 1, wherein the FLCHCP and SLCHCP comprise different light chains.

11. The antibody of claim 1, wherein the antibody is free of (a) non-naturally occurring intramolecular cysteine-cysteine disulfide bonds; (b) protuberance and cavity modifications in the multimerization domain; (c) artificial hydrophilic or hydrophobic sequence modifications comprising two or more contiguous amino acid residue substitutions; or (d) any combination of (a)-(c).

12. The antibody of claim 1, wherein the antibody is free of any linkage to one or more additional antibody molecules or fragments by covalent linkage.

13. A method of producing a bispecific antibody comprising contacting
(i) a first light chain protein (“FLCP”);
(ii) a first heavy chain protein (“FHCP”) comprising the substitutions K253E, D282K, and K322D;
wherein the FLCP and FHCP are capable of forming a FLCHCP having specificity for a first target;
(iii) a second light chain protein (“SLCP”); and
(iv) a second heavy chain protein (“SHCP”) comprising the substitutions K253E, D282K, and K322D;
wherein the SLCP and SHCP are capable of forming a SLCHCP having specificity for a second target,
under conditions suitable for the formation of a bispecific antibody comprising the FLCHCP and SLCHCP, wherein either the FLCHCP or SLCHCP comprises a light chain having the substitution E15K and a heavy chain comprising the substitution K96E.

14. A method of producing a bispecific antibody comprising:
(a) identifying pairs of amino acid residues involved in intramolecular ionic interactions in a wild-type tetrameric antibody molecule of the isotype in an organism,
(b) preparing (i) FLCP and FHCP capable of forming a FLCHCP comprising at least some substitutions of amino acid residues involved in such wild-type antibody intramolecular interactions and having specificity for a first target and (ii) SLCP and SHCP capable of forming a SLCHCP having specificity for a second target and comprising an amino acid sequence complementary to the first light chain-heavy chain pair in terms of such intramolecular ionic interactions, the FLCHCP and SLCHCP collectively comprising substitution of a sufficient number of amino acid residues involved in such wild-type antibody intramolecular interactions that bispecific tetramers comprising the FLCHCP and SLCHCP form more frequently than molecules comprising only the FLCHCP or SLCHCP when the FLCP, FHCP, SLCP, and SHCP are permitted to mix, and
(c) mixing the FLCP, FHCP, SLCP, and SHCP or the FLCHCP and SLCHCP under suitable conditions so as to produce a bispecific antibody.

15. A bispecific antibody comprising a FLCHCP having specificity for a first target and a sufficient number of substitutions in its heavy chain constant domain with respect to a corresponding wild-type antibody of the same isotype to significantly reduce the formation of first heavy chain-first heavy chain dimers and a SLCHCP comprising a heavy chain having a sequence that is complementary to the sequence of the FLCHCP heavy chain sequence with respect to the formation of intramolecular ionic interactions, wherein the FLCHCP or the SLCHCP comprises a substitution in the light chain and complementary substitution in the heavy chain that reduces the ability of the light chain to interact with the heavy chain of the other LCHCP.


SEQUENCE LISTING (132 sequences)

SEQ ID NO: 1 - protein, 137 aa, Artificial
  synthetic
MDRLTSSFLLLIVPAYVLSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 2 - protein, 131 aa, Artificial
  synthetic
METDTILLWVLLLWVPGSTGDTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 3 - protein, 128 aa, Artificial
  synthetic
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 4 - protein, 140 aa, Artificial
  synthetic
MGWSLILLFLVAVATRVLSQVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 5 - protein, 125 aa, Artificial
  synthetic
MAQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTLVTVSTG

SEQ ID NO: 6 - protein, 110 aa, Artificial
  synthetic
MAQIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKVEIKRK

SEQ ID NO: 7 - protein, 32 aa, Artificial
  synthetic
QVQLQESGPSLVKPGASVKIVCKASGYTFTRL

SEQ ID NO: 8 - protein, 55 aa, Artificial
  synthetic
ADGVPSRFSGSGSGTQFSLKINRLQPEDFGNYYCQHFWSTPWTFGGGTKLEIKRA

SEQ ID NO: 9 - protein, 120 aa, Artificial
  synthetic
QVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVS

SEQ ID NO: 10 - protein, 106 aa, Artificial
  synthetic
DIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELK

SEQ ID NO: 11 - protein, 10 aa, Artificial
  synthetic
GFNIKEYYMH

SEQ ID NO: 12 - protein, 17 aa, Artificial
  synthetic
LIDPEQGNTIYDPKFQD

SEQ ID NO: 13 - protein, 8 aa, Artificial
  synthetic
DTAAYFDY

SEQ ID NO: 14 - protein, 11 aa, Artificial
  synthetic
RASRDIKSYLN

SEQ ID NO: 15 - protein, 7 aa, Artificial
  synthetic
YATSLAE

SEQ ID NO: 16 - protein, 9 aa, Artificial
  synthetic
LQHGESPWT

SEQ ID NO: 17 - protein, 228 aa, Artificial
  synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 18 - protein, 120 aa, Artificial
  synthetic
EVQVQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSS

SEQ ID NO: 19 - protein, 117 aa, Artificial
  synthetic
QIQLVQSGPELKKPGETVKISCKASGYTFTNYGMNWVKQAPGESLKWMGWLNTNTGEPTYAEDFKGRFAFSLGTSASTAYLRINNVKDEDTATYFCARWGRDDVGYWGQGTTLIVSS

SEQ ID NO: 20 - protein, 20 aa, Artificial
  synthetic
EVQLVESGGGLVQPKGSLKL

SEQ ID NO: 21 - protein, 214 aa, Artificial
  synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 113 aa, Artificial
  synthetic
DVLMTQTPLSLPVSLGDQASISCRSGQSIVHSNGNTYLEWYLQKPGQSPKLLIYRVSNRFSGVPDRFSGSGSGSDFTLKISRVEAEDLGVYYCFQYSHVPWTFGGGTKLEIKR

SEQ ID NO: 23 - protein, 114 aa, Artificial
  synthetic
DIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKR

SEQ ID NO: 24 - protein, 20 aa, Artificial
  synthetic
DIVMTQSQKFMSTSVVDRIS

SEQ ID NO: 25 - protein, 119 aa, Artificial
  synthetic
QVQLQESGPELVRPGASVKMSCKASGYTFTTYWIHWMKQRPGQGLQWIGMIDPSNSETRLNQNFRDKATLSVDKSSNKAYMQLSSLTSEDSAIYYCARWDYGSGHFDYWGQGTTVTVSS

SEQ ID NO: 26 - protein, 119 aa, Artificial
  synthetic
QVQLQESGPELVKPGALVKISCKASGYTFTSYWMHWVKQRPGQGLEWIGEIDPSDSYTNYNQKFKGKATLTVDKSSNTAYMQLSSLTSEDSAVYYCARSDYGSSHFDYWGQGTTVTVSS

SEQ ID NO: 27 - protein, 112 aa, Artificial
  synthetic
DIELTQSPASLAVSLGQRATISCRASESVDNFGISFMNWFQQKPGQPPKLLIYGASNQGSGVPARFSGSGSGTDFSLNIHPLEEDDTAMYFCQQSKEVPLTFGAGTKLEIKR

SEQ ID NO: 28 - protein, 119 aa, Artificial
  synthetic
EVQLQQSGAELVKPGASVKLSCKASGYTFTSYWMHWVKQRPGQGLEWIGEIDPSDSYTNYNQKFKGKATLTVDKSSSTAYMQLSSLTSEDSAVYYCARSDYGSSHFDYWGQGTTVTVSS

SEQ ID NO: 29 - protein, 113 aa, Artificial
  synthetic
DIELTQSPASLAVSLGQRATISCRASESVDNFGISFMNWFQQKPGQPPKLLIYGASNQGSGVPARFSGSGSGTDFSLNIHPLEEDDTAMYFCQQSKEVPLTFGAGTKLELKRA

SEQ ID NO: 30 - protein, 118 aa, Artificial
  synthetic
EVKLQQSGPELVKPGASVKMSCKASGYAFISFVMHWVKQKPGQGLEWIGFINPYNDGTKYNEKFKDKATLTSDKSSSTAYMELSSLTSEDSAVYYCASGDYDRAMDYWGQGTTVTVSS

SEQ ID NO: 31 - protein, 109 aa, Artificial
  synthetic
DIELTQSPTTMAASPGEKITITCSASSSISSNYLHWYQQKPGFSPKLLIYRTSNLASGVPARFSGSGSGTSYSLTIGTMEAEDVATYYCQQGSSIPRTFGGGTKLEIKR

SEQ ID NO: 32 - protein, 11 aa, Artificial
  synthetic
RASQSVSSYLA

SEQ ID NO: 33 - protein, 7 aa, Artificial
  synthetic
DSSNRAT

SEQ ID NO: 34 - protein, 9 aa, Artificial
  synthetic
LQHNTFPPT

SEQ ID NO: 35 - protein, 10 aa, Artificial
  synthetic
GFTFSSYSMN

SEQ ID NO: 36 - protein, 17 aa, Artificial
  synthetic
SISSSSSYIYYADSVKG

SEQ ID NO: 37 - protein, 7 aa, Artificial
  synthetic
VTDAFDI

SEQ ID NO: 38 - protein, 11 aa, Artificial
  synthetic
RASQGISSRLA

SEQ ID NO: 39 - protein, 7 aa, Artificial
  synthetic
AASSLQT

SEQ ID NO: 40 - protein, 9 aa, Artificial
  synthetic
QQANRFPPT

SEQ ID NO: 41 - protein, 10 aa, Artificial
  synthetic
GFTFSSYSMN

SEQ ID NO: 42 - protein, 17 aa, Artificial
  synthetic
SISSSSSYIYYADSVKG

SEQ ID NO: 43 - protein, 7 aa, Artificial
  synthetic
VTDAFDI

SEQ ID NO: 44 - protein, 14 aa, Artificial
  synthetic
AGTTTDLTYYDLVS

SEQ ID NO: 45 - protein, 7 aa, Artificial
  synthetic
DGNKRPS

SEQ ID NO: 46 - protein, 10 aa, Artificial
  synthetic
NSYVSSRFYV

SEQ ID NO: 47 - protein, 10 aa, Artificial
  synthetic
GFTFSSYSMN

SEQ ID NO: 48 - protein, 17 aa, Artificial
  synthetic
SISSSSSYIYYADSVKG

SEQ ID NO: 49 - protein, 7 aa, Artificial
  synthetic
VTDAFDI

SEQ ID NO: 50 - protein, 13 aa, Artificial
  synthetic
SGSTSNIGTNTAN

SEQ ID NO: 51 - protein, 7 aa, Artificial
  synthetic
NNNQRPS

SEQ ID NO: 52 - protein, 12 aa, Artificial
  synthetic
AAWDDSLNGHWV

SEQ ID NO: 53 - protein, 10 aa, Artificial
  synthetic
GGTFSSYAIS

SEQ ID NO: 54 - protein, 18 aa, Artificial
  synthetic
GGIIPIFGTANYAQKFQG

SEQ ID NO: 55 - protein, 16 aa, Artificial
  synthetic
GYDYYDSSGVASPFDY

SEQ ID NO: 56 - protein, 108 aa, Artificial
  synthetic
DIKMTQSPSFLSASVGDRVTLNCKASQNIDKYLNWYQQKLGESPKLLIYNTNNLQTGIPSRFSGSGSGTDFTLTISSLQPEDVATYFCLQHISRPRTFGTGTKLELKR

SEQ ID NO: 57 - protein, 120 aa, Artificial
  synthetic
EVKLLESGGGLVPGGSMRLSCAGSGFTFTDFYMNWIRQPAGKAPEWLGFIRDKAKGYTTEYNPSVKGRFTISRDNTQNMLYLQMNTLRAEDTATYYCAREGHTAAPFDYWGQGVMVTVSS

SEQ ID NO: 58 - protein, 108 aa, Artificial
  synthetic
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKR

SEQ ID NO: 59 - protein, 122 aa, Artificial
  synthetic
QSVQLQESGPGLVRPSQTLSLTCTVSGSTFSDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 60 - protein, 139 aa, Artificial
  synthetic
MNKAMRBPMEKDTLLLWVLLLWVPGSTGDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 61 - protein, 135 aa, Artificial
  synthetic
MGWSWIFLFLLSGTAGVHSEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSS

SEQ ID NO: 62 - protein, 112 aa, Artificial
  synthetic
DIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 63 - protein, 115 aa, Artificial
  synthetic
QVQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGFAYWGQGTTVTVS

SEQ ID NO: 64 - protein, 121 aa, Artificial
  synthetic
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 65 - protein, 113 aa, Artificial
  synthetic
DVVVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 66 - protein, 121 aa, Artificial
  synthetic
EVQLVQSGAEVKKPGASVKVSCKASGYRFTNYWIHWVRQAPGQGLEWIGGINPGNNYATYRRNLKGRVTMTADTSTSTVYMELSSLRSEDTAVYYCTREGYGNYGAWFAYWGQGTLVTVSS

SEQ ID NO: 67 - protein, 113 aa, Artificial
  synthetic
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 68 - protein, 121 aa, Artificial
  synthetic
EVQLVQSGAEVKKPGASVKVSCKASGYRFTNYWIHWVRQAPGQGLEWIGGINPGNNYATYRRNLKGRATLTADTSTSTVYMELSSLRSEDTAVYYCTREGYGNYGAWFAYWGQGTLVTVSS

SEQ ID NO: 69 - protein, 113 aa, Artificial
  synthetic
DVVVTQTPLSLPVSFGDQVSISCRSSQSLANSYGNTFLSWYLHKPGQSPQLLIYGISNRFSGVPDRFTGSGSGTDFTLKISTIKPEDLGMYYCLQGTHQPYTFGGGTKLEIKR

SEQ ID NO: 70 - protein, 121 aa, Artificial
  synthetic
EVQLQQSGTVLARPGASVKMSCKASGYRFTNYWIHWVKQRPGQGLEWIGGINPGNNYTTYKRNLKGKATLTAVTSASTAYMDLSSLTSEDSAVYYCTREGYGNYGAWFAYWGQGTLVTVSS

SEQ ID NO: 71 - protein, 113 aa, Artificial; identical to SEQ ID NO: 69
  synthetic

SEQ ID NO: 72 - protein, 107 aa, Artificial
  synthetic
DIQMTQSPASLSASVGETVTFTCRASENIYSYLAWYQQKQGKSPQLLVHDAKTLAEGVPSRFSGGGSGTQFSLKINTLQPEDFGTYYCQHHYGNPPTFGGGTKLEIK

SEQ ID NO: 73 - protein, 117 aa, Artificial
  synthetic
QVQLKQSGPGLVQPSQSLSITCTVSGFSLTTFGVHWVRQSPGKGLEWLGVIWRSGITDYNVPFMSRLSITKDNSKSQVFFKLNSLQPDDTAIYYCAKNDPGTGFAYWGQGTLVTVSA

SEQ ID NO: 74 - DNA, 45 bp, Artificial
  synthetic
ctaatacgactcactatagggcaagcagtggtatcaacgcagagt

SEQ ID NO: 75 - DNA, 21 bp, Artificial
  synthetic
ctaatacgactcactataggg

SEQ ID NO: 76 - DNA, 22 bp, Artificial
  synthetic
tcatttacccggggacagggag

SEQ ID NO: 77 - DNA, 23 bp, Artificial
  synthetic
tcatttacccagagacagggaga

SEQ ID NO: 78 - DNA, 45 bp, Artificial; identical to SEQ ID NO: 74
  synthetic

SEQ ID NO: 79 - DNA, 21 bp, Artificial
  synthetic
ctaatacgactcactataggg

SEQ ID NO: 80 - DNA, 25 bp, Artificial
  synthetic
ctaacactctcccctgttgaagctc

SEQ ID NO: 81 - DNA, 16 bp, Artificial
  synthetic
gtaaaacgacggccag

SEQ ID NO: 82 - DNA, 17 bp, Artificial
  synthetic
caggaaacagctatgac

SEQ ID NO: 83 - DNA, 22 bp, Artificial
  synthetic
gcctggtcgagggcttctatcc

SEQ ID NO: 84 - DNA, 22 bp, Artificial
  synthetic
cctcccgtgctgaaatccgacg

SEQ ID NO: 85 - DNA, 33 bp, Artificial
  synthetic
ccactacacgcaggacagcctctccctgtcccc

SEQ ID NO: 86 - DNA, 32 bp, Artificial
  synthetic
cccagcaacaccaaggtggacgagagagttga

SEQ ID NO: 87 - DNA, 29 bp, Artificial
  synthetic
tgcccccatcccggaagaaaatgaccaag

SEQ ID NO: 88 - DNA, 31 bp, Artificial
  synthetic
tccttcttcctctatagcgatctcaccgtgg

SEQ ID NO: 89 - DNA, 30 bp, Artificial
  synthetic
catcttcccgccatctgataagcagttgaa

SEQ ID NO: 90 - DNA, 25 bp, Artificial
  synthetic
gcctggtcgaaggcttctaccccag

SEQ ID NO: 91 - DNA, 25 bp, Artificial
  synthetic
ctcccgtgctgaaatccgacggctc

SEQ ID NO: 92 - DNA, 24 bp, Artificial
  synthetic
actacacacaggacagcctctccc

SEQ ID NO: 93 - DNA, 22 bp, Artificial
  synthetic
tcaactctctcgtccaccttgg

SEQ ID NO: 94 - DNA, 25 bp, Artificial
  synthetic
caaggtggacgagagagttgagtcc

SEQ ID NO: 95 - DNA, 25 bp, Artificial
  synthetic
cccatcccagaagaagatgaccaag

SEQ ID NO: 96 - DNA, 25 bp, Artificial
  synthetic
ctctacagcgatctaaccgtggaca

SEQ ID NO: 97 - DNA, 24 bp, Artificial
  synthetic
gctagcaccaagggcccatccgtc

SEQ ID NO: 98 - DNA, 42 bp, Artificial
  synthetic
gcgcagatcttcatttacccggggacagggagaggctgtcct

SEQ ID NO: 99 - DNA, 31 bp, Artificial
  synthetic
cggccgtacggtggctgcaccatctgtcttc

SEQ ID NO: 100 - DNA, 34 bp, Artificial
  synthetic
gcgctctagactaacactcattcctgttgaagct

SEQ ID NO: 101 - DNA, 32 bp, Artificial
  synthetic
gcgcagatcttcatttacccggggacagggag

SEQ ID NO: 102 - DNA, 40 bp, Artificial
  synthetic
gaagatcttcatttacccagagacagggagaggctgtcct

SEQ ID NO: 103 - DNA, 32 bp, Artificial
  synthetic
gaagatcttcatttacccagagacagggagag

SEQ ID NO: 104 - DNA, 38 bp, Artificial
  synthetic
gcgcaagcttgccaccatggaagccccagctcagcttc

SEQ ID NO: 105 - DNA, 32 bp, Artificial
  synthetic
gcgccgtacgtttgatctccaccttggtccct

SEQ ID NO: 106 - DNA, 32 bp, Artificial
  synthetic
ggccgcggccgcaccatggagtttgggctgag

SEQ ID NO: 107 - DNA, 28 bp, Artificial
  synthetic
gccggctagctgaggagacggtgaccag

SEQ ID NO: 108 - DNA, 38 bp, Artificial; identical to SEQ ID NO: 104
  synthetic

SEQ ID NO: 109 - DNA, 30 bp, Artificial
  synthetic
gcgccgtacgtttgatctccagcttggtcc

SEQ ID NO: 110 - DNA, 30 bp, Artificial
  synthetic
gcggccgccatggactggacctggaggttc

SEQ ID NO: 111 - DNA, 31 bp, Artificial
  synthetic
gccggctagctgaggagacggtgaccgtggt

SEQ ID NO: 112 - DNA, 32 bp, Artificial
  synthetic
gcggccgccatggctagcaccaagggcccatc

SEQ ID NO: 113 - DNA, 32 bp, Artificial; identical to SEQ ID NO: 101
  synthetic

SEQ ID NO: 114 - DNA, 33 bp, Artificial
  synthetic
ctcacacagcgccaccggcgccagcacctgaac

SEQ ID NO: 115 - DNA, 33 bp, Artificial
  synthetic
ggtcccccagcgccatcagcgccagcacctgag

SEQ ID NO: 116 - protein, 330 aa, Homo sapiens [sequence omitted]

SEQ ID NO: 117 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 120 - protein, 326 aa, Rattus norvegicus
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 121 - protein, 329 aa, Mus musculus
TTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 122 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 123 - protein, 335 aa, Mus musculus
AKTTAPSVYPLVPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPHQRVPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 124 - protein, 322 aa, Rattus norvegicus
AETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 125 - protein, 336 aa, Mus musculus
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 126 - protein, 333 aa, Rattus norvegicus
AQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 127 - protein, 329 aa, Rattus norvegicus
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 128 - protein, 330 aa, Homo sapiens [sequence omitted]

SEQ ID NO: 129 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 130 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 131 - protein, 290 aa, Homo sapiens
QMQGVNCTVSSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVQVHNAKTKPREQQFNSTFRVVSVLTVLHQNWLDGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 132 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK